(19) United States
(12) Patent Application Publication
(10) Pub. No.: US 2021/0400211 A1
(43) Pub. Date: Dec. 23, 2021

(54) MULTI-MODAL MOBILE THERMAL IMAGING SYSTEM

(71) Applicant: Woundtech, Hollywood, FL (US)

(72) Inventors: Jeffrey L. Galitz, Bal Harbor, FL (US); Minghsun Liu, Los Angeles, CA (US)

(21) Appl. No.: 17/304,592

(22) Filed: Jun. 23, 2021

Related U.S. Application Data
(60) Provisional application No. 63/042,957, filed on Jun. 23, 2020.

(51) Int. Cl. H04N 5/33; G02B 5/20; G06T 7/00; G06K 9/62

(52) U.S. Cl. CPC H04N 5/332; G02B 5/208; G06T 7/0012; G06T 2207/10064; G06T 2207/30024; G06T 2207/20024; G06T 2207/10048; G06K 9/628

(57) ABSTRACT

A mobile imaging system can include a camera system configured to capture images in a visible, a near-infrared, and an infrared light spectrum, a filter assembly including at least two optical filters, an illumination module activatable to emit light, and a computer system including processing circuitry configured to perform operations including control the camera system to collect multispectral imaging data of biological tissue associated with an injury or ailment of a patient and process the multispectral imaging data to assess the injury or ailment; a battery arranged to power the mobile imaging system, and a housing encompassing the processing circuitry and the battery.

### CLAIM OF PRIORITY

This patent application claims the benefit of priority of U.S. Provisional Patent Application Ser. No. 63/042,957 entitled “MULTI-MODAL MOBILE THERMAL IMAGING SYSTEM” filed on Jun. 23, 2020, which is hereby incorporated by reference herein in its entirety.

### TECHNICAL FIELD

The present disclosure relates generally to the use of multispectral imaging in the diagnosis and treatment of various medical conditions. More particularly, the present disclosure relates to use of a mobile device, such as smartphone, to perform multispectral imaging of tissue associated with a wound, injury, or other ailment of a patient.

### BACKGROUND

Some medical conditions, such as chronic wounds or illnesses, can be difficult to accurately diagnose and treat. For many patients, the diagnosis and treatment of such conditions is often complex and expensive due to a wide variation between clinical assessments of the condition. For example, an assessment of a chronic wound or illness made at one point-of-care location can often vary significantly from an assessment of the same, or similar, wound illness made at another point-of-care location. Such variation is often due to the wide range of training and clinical experience between different healthcare providers.

Diagnosis and treatment of wounds is also difficult because wounds tend to heal over periods of time, and therefore tracking a progression of the wound condition is valuable in treating wounds. It is difficult and expensive to have wound patients examined at a health care location repeatedly to track the progression of the wound and the efficacy of the treatment.

There is a need in the art for a system to monitor wounds, infections, and other medical conditions of patients over time. There is also a need in the art for a system which does not require a patient frequent office visits to track the progression of a wound.

### SUMMARY

The present subject matter provides, among other things, methods and apparatus for a thermal imaging system for wound and other medical condition imaging that is relatively inexpensive, portable, and which will enhance doctors in diagnosing and treating wounds, infections, and other medical conditions. In various embodiments, the present subject matter allows for remote monitoring of wounds and other medical conditions over time. In various applications, the present subject matter allows for a health care provider to review wound and other medical conditions from remote, saving the cost and time of patient travel and office visits. Other advantages of the present subject matter will be apparent to those of skill in the art upon reading and understanding this patent application.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1-2 illustrate front and rear isometric views, respectively, of an example an imaging system, according to various embodiments of the present subject matter.

FIG. 3 illustrates a schematic view of signal communication between components of an example imaging system, according to various embodiments of the present subject matter.

FIG. 4 illustrates a partially exploded view of an example imaging system, according to various embodiments of the present subject matter.

FIG. 5 illustrates a rear isometric view of an example imaging system, according to various embodiments of the present subject matter.

FIGS. 6-8 illustrate front, rear, and side views, respectively, of an example of an imaging system, according to various embodiments of the present subject matter.

FIG. 9 illustrates a front isometric view of an example filter assembly, according to various embodiments of the present subject matter.

FIGS. 10-12 illustrate spectral graphs of various optical filters of an example filter assembly, according to various embodiments of the present subject matter.

FIG. 13 illustrates a front isometric view of an example illumination module, according to various embodiments of the present subject matter.

FIG. 14 illustrates a front isometric view of an example controller of an example illumination module, according to various embodiments of the present subject matter.

FIGS. 15-17 illustrate spectral graphs of various wavelengths of light emission of an example illumination module, according to various embodiments of the present subject matter.

FIG. 18 illustrates a flowchart of an example method of assessing an injury or ailment of a patient using an example imaging system.

FIG. 19 illustrates a graph comparing example Isosbestic characteristics of oxyhemoglobin (HbO2) and deoxyhemoglobin (Hb).

FIG. 20 illustrates flowchart of an example pathway of recording various signals usable in a method of assessing an injury or ailment of a patient using an example imaging system.

### DETAILED DESCRIPTION

The present subject matter provides, among other things, methods and apparatus for a thermal imaging system for wound imaging that are relatively inexpensive, portable, and which will enhance doctors' abilities to diagnose and treat wounds, infections, and other medical conditions, according to various embodiments of the present subject matter.

Various abnormal physical characteristics or parameters of biological tissue, such as associated with an injury, chronic wound, disease, or infection, are known to cause, contribute to, or delay the healing of various medical conditions. For example, physical characteristics including edema or swelling, tissue oxygenation, tissue perfusion, bacterial load or bioburden, a measurable wound area, or a measurable wound volume, among others, can be analyzed, such as via quantification or classification, to enable a physician to diagnose or treat a medical condition of a patient. Multispectral imaging is can be an effective technique of measuring, quantifying, or classifying physical characteristics of a patient's tissue; and includes collecting both images and spectroscopic data of patient tissue to advantageously obtain both spatial and spectral information associated with a medical condition.

For example, when only traditional imaging is used (e.g. visible light spectrum photos or videos) to assess a patient, certain characteristics indicative of an abnormal medical condition, such as skin color or temperature, are not visible. Spectroscopic data collection can help to address such issues by measuring compositional changes in affected tissue by capturing an entire spectrum of a tissue area within a certain frequency or wavelength. However, significant spatial information about the affected tissue, such as edema, or a measurable wound area or volume, cannot be collected or assessed using only spectroscopic data. As such, collecting and assessing both spatial and spectroscopic data can significantly improve the diagnosis and treatment of many medical conditions.

Generally, multispectral imaging can include collecting and analyzing images in two, three, four, or five relatively noncontiguous, or widely spaced, spectral bandwidths. For example, multispectral imaging of an affected tissue can be performed using a combination of visible, near infrared, or infrared cameras. However, commercially available systems or devices operable to perform such multispectral imaging are often expensive, complex, and limited in portability. Additionally, such systems or devices can be limited to assessing an injury or ailment of an individual patient in isolation, or during only one point in time. As such, a provider may have to manually, or subjectively, compare collected imaging data of a patient to imaging data of the same patient, or other patients, collected at previous or former points in time to improve the sensitivity and specificity, and thereby the accuracy, of an assessment or diagnosis.

The present subject matter can help to address these issues, among others, such as by providing an easily portable, relatively inexpensive, multispectral, and multi-modal imaging system. The imaging system of the present disclosure can be capable of capturing and processing images of biological tissue associated with an injury or ailment of a patient in various spectral ranges. For example, the imaging system can be smartphone based and comprised of commercially available components to lower the cost and improve portability over existing and dedicated multispectral imaging systems. In some examples, an example multispectral imaging system can be realized as smartphone connected to any of an infrared camera, a near-infrared camera, a filter assembly including optical (e.g., spectral filters), or illumination module operable to emit light in various spectral frequencies or wavelengths.

Further, the imaging system of the present disclosure can be configured to enable users, such as primary point-of-care or general practitioners, to develop improved treatment strategies by using new clinical diagnostic pathways or methods to improve the sensitivity and the specificity of an assessment of diagnosis of a medical condition, and thereby reduce the present and widespread variation in the clinical assessments of many wounds, injuries, or ailments. For example, an imaging system according to the present disclosure can include a mobile application or other software running on processing circuitry of a smartphone operable to quantify or classify multispectral imaging data by objectively comparing such data to previously collected data associated with similar injuries or wounds of the patient, or of other patients, such as stored on a remote imaging database. Accordingly, the imaging system of present disclosure can significantly reduce the cost of and improve the accuracy of assessment, diagnosis, and treatment of various medical conditions.

While the above overview discusses examples generally usable by a primary point-of-care provider or general practitioner, discussion of the following systems, devices, or methods is also applicable for use by other healthcare practitioners, such as surgical oncologists, podiatrists, plastic surgeons, hospitalists, researchers, or other point-of-care locations, such as in emergency rooms, lymphedema clinics, rural clinics, clinical trials, hospital-based operating rooms, or general surgeries. The above overview is intended to provide an overview of present subject matter and is not intended to provide an exclusive or exhaustive explanation of the present subject matter.

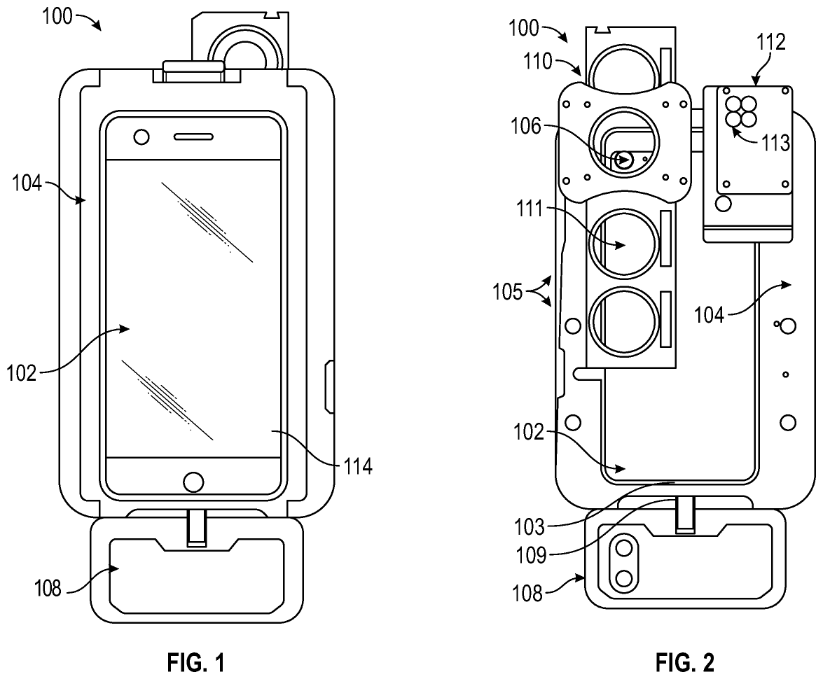

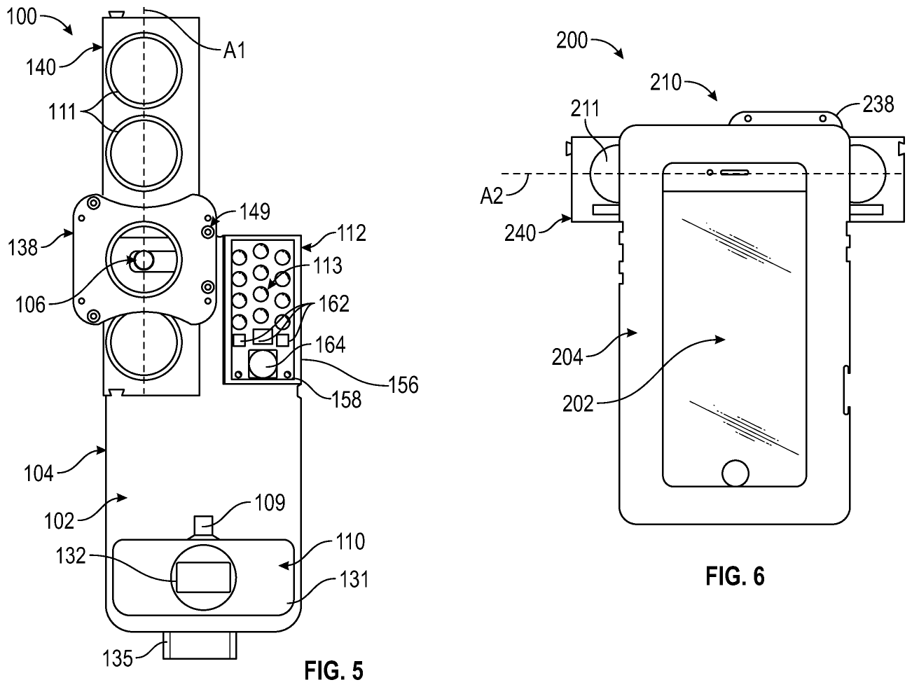

FIGS. 1-2 illustrate front and rear isometric views, respectively, of an example an imaging system 100. FIGS. 1-2 are discussed below concurrently. In some examples, the imaging system 100 can include a mobile device 102, a housing 104, a camera system 105, and an illumination module 112. The mobile device 102 can be a variety of computer systems, such as including any of a smartphone, electronic tablet, laptop computer, or other generally portable electronic devices. In one example, the mobile device 102 can be an iPhone® of any current or former model. The mobile device 102 can be internet-enabled, such as to transmit and retrieve images, videos, or other data from a remote database or data warehouse, such as a cloud service. In some examples, such has shown in FIGS. 1-2, the housing 104 can be a Beastcage® made by Beastgrip of Des Plaines, Ill. In other examples, such as shown in FIGS. 5-8, the housing 104 can be custom or proprietary housing configured to accept a particular or specific mobile device 102, such as sized and shaped to completely, or partially, encompass the mobile device 102. The housing 104 can be made from any of various materials include, but not limited, to, metals, plastics, composites, silicone, or rubber.

In various embodiments, the camera system 105 (FIG. 2) includes a first camera 106, a second camera 108, and a filter assembly 110. In some examples, the first camera 106 includes a camera integrated into the mobile device 102. The first camera 106 can be configured to capture traditional or otherwise conventional imaging data (e.g., an image or video in a visible light spectrum). In some examples, the first camera 106 is configured, such as via one or more modifications, to capture near-infrared images or video using the mobile device 102. The second camera 108, in some examples, can be a camera externally connected to the mobile device 102. For example, the second camera 108 can be in electrical communication with the mobile device 102, such as to be controlled by and receive power from, the mobile device 102 via an electrical connector 109 extending therefrom. In such an example, the electrical connector 109 can extend into or otherwise engage a port 103, or other device interface, of the mobile device 102. In some examples, the second camera 108 can be configured to capture images in an infrared light spectrum or in a near-infrared light spectrum.

The filter assembly 110 (FIG. 2) can be, in some examples, a standalone mechanical device including a plurality of optical (e.g., spectral) filters 111 (FIG. 2). The filter assembly 110 can be connected to the housing 104 by any of various means, such as via fasteners or adhesives. The filter assembly 110 can be operable or otherwise configurable to allow a user to selectively position any of the plurality of optical filters 111 proximally to (e.g., in front of) the first camera 106. For example, a portion of the filter assembly 110 can be translatable by a user along an axis defined by the filter assembly 110 to sequentially position the plurality of filters 111 in front of the first camera 106 to capture images in various independent and non-contiguous spectral ranges. The filter assembly 110 can thereby enable the imaging system 100 to selectively capture multispectral images in additional multispectral ranges (e.g., wavelengths) beyond which the first camera 106 and the second camera 108 could otherwise collect.

The illumination module 112 (FIG. 2) can generally be an illumination device configured to emit light in various, such as independent or non-contiguous multispectral ranges. In various embodiments, the illumination module 112 is connected to the housing 104 by any of various means, such as via fasteners or adhesives. The illumination module 112 can include a plurality of light emitters 113 (FIG. 2), such as light emitting diodes (LEDs). The light emitters may be grouped, for example, to be operable or otherwise activatable to sequentially and thus independently, emit light in different frequencies or wavelengths. In some examples, the light emitters 113 are configured, such as by a user, to emit light in various independent and non-contiguous spectral ranges corresponding to such spectral ranges of the optical filters 111.

The mobile device 102 may further include a user interface 114 (FIG. 1). The user interface 114 may include various input or output devices, such as a touch screen of a smartphone (e.g., the mobile device 102). As such, the user interface 114 may be user operable to control various operations of at least devices in electrical communication with processing circuitry 116 (schematically illustrated in FIG. 2) of the mobile device 102. For example, the user interface 114 can receive one or more user inputs to cause the first camera 106, the second camera 108, or the illumination module 112 to activate to collect or multispectral imaging data, such as based on a particular wound, injury, or ailment of a patient. In such an example, the processing circuitry 116 can run a mobile application or other software configured to implement various operations of the imaging system 100.

In the operation of some examples of the imaging system 100, a user can first configure the imaging system 100, such as via one or more user inputs to any of the user interface 114 of the mobile device 102, the first camera 106, the second camera 108, the filter assembly 110, or the illumination module 112, such as to configure the imaging system 100 to perform multispectral imaging based on a medical condition of a patient upon activation or operation. A user can then activate or otherwise operate the imaging system 100 to capture and collect multispectral imaging data associated with the medical condition of the patient, such as including collecting both visible light spectrum images and near infrared or infrared images. During such operation, in some examples, a user can further position any of the optical filters 111 in a position proximal to the first camera 106, or manually engage features of the illumination module 112, such as to help collect imaging data in a wider or otherwise additional range of frequencies or wavelengths.

In some examples, the collected multispectral imaging data can include images or video collected in two to five spectral ranges, such as aggregated into a single data set in the form of a three-dimensional multispectral data cube. The imaging system 100 can then implement analysis of the collected multispectral imaging data via software running on the processing circuitry 116 of the mobile device 102, such as automatically upon collection or manually via one or more user inputs to the user interface 114. Such analysis can include quantifying or classifying various physical characteristics or parameters of imaged biological tissue. The imaging system 100 can then output any resulting data, such by displaying the data on the user interface 114 of the mobile device 102. A user, such as a physician, can thereby view and consider the data to make an assessment or diagnosis of a wide variety of medical conditions, such as from standalone injuries to chronic wounds or infections, based on, for example, quantified or classified tissue edema or swelling, tissue oxygenation, tissue perfusion, bacterial load, bioburden, wound area, wound volume, or still further physical characteristics of imaged patient tissue.

Finally, as the mobile device 102 can be internet enabled, the processing circuitry 116 of the mobile device 102 can compare such analyzed data against raw or analyzed data collected from the same patient at a previous or former point in time, or from other patients. Such data can be, for example, stored on a remote imaging database or cloud service, and can used to improve the accuracy of an assessment or diagnosis, such by averaging or otherwise referencing the previously collected or analyzed data. Still further, the previously collected or analyzed data can be used by various healthcare providers to establish a library of standardized values or ranges associated with a particular medical condition, such as to establish new pathways to diagnose medical conditions that many be otherwise difficult to accurately assess. In view of the above, the imaging system 100 can provide a number of benefits to both a patients and a user, such as, but not limited to, reducing the cost of an assessment for a patient, increasing the accuracy of and reducing variation between assessments of various medical conditions, and improving both the portability and accessibility of a multispectral imaging system usable to diagnose and treat medical conditions.

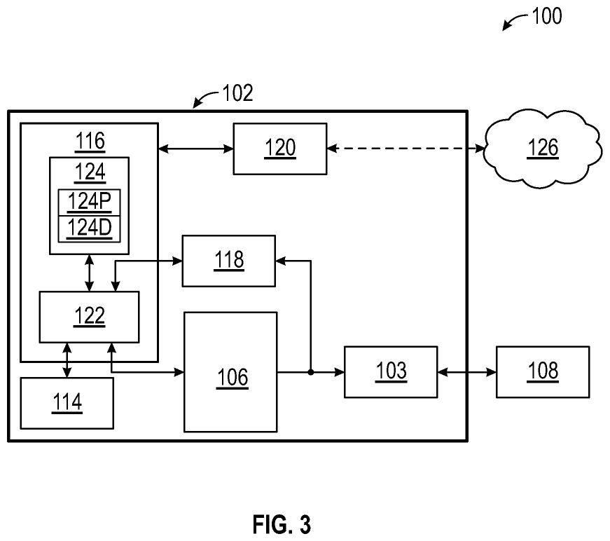

FIG. 3 illustrates an example signal communication schematic of several components of the imaging system 100. FIG. 3 is discussed with reference to the example imaging system 100 shown in FIG. 1 above. In some examples, signal communication between components of the imaging system 100 can be realized using the elements shown in FIG. 3. In other examples, the imaging system 100 can include other elements in signal communication with any of the mobile device 102, the first camera 106, or the second camera 108, such as in an example where the illumination module 112 is internet-enabled or is otherwise electrical communication with the mobile device 102. As illustrated in FIG. 3, the mobile device 102 can include the port 103, the first camera 106, the user interface 114, the processing circuitry 116, a power source 118, and a communications module 120.

In some examples, the processing circuitry 116 can include at least a processor 122 and a memory 124. In some examples, the processor 122 includes a timer and/or a clock. In other examples, the timer and/or clock can be an element of, or included in a device separate from, the processor 122. The processor 122 can include a hardware processor, such as a central processing unit (CPU), a graphics processing unit (GPU), a hardware processor core, or any combination thereof. The processor 122 can include any of a microprocessor, a control circuit, a digital signal processor (DSP), an application specific integrated circuit (ASIC), a field-programmable gate array (FPGA), or other equivalent discrete or integrated logic circuitry.

In some examples, the memory 124 includes computer-readable storage media. In some examples, a computer-readable storage media can include a non-transitory medium. The term “non-transitory” can indicate that the storage medium is not embodied in a carrier wave or a propagated signal. In some examples, a non-transitory storage medium can store data that can, over time, change (e.g., in RAM or cache). In some examples, the memory 124 can be a temporary memory, such as meaning that a primary purpose of the memory 124 is not a long-term storage. In some examples, the memory 124 can be described as volatile memory, meaning that the memory 124 does not maintain stored contents when power to the mobile device 102 is turned off. Examples of volatile memories can include random access memories (RAM), dynamic random-access memories (DRAM), static random-access memories (SRAM), and other forms of volatile memories.

In some examples, the memory 124 can include one or more computer-readable storage media. In some examples, the memory 124 can be configured to store larger amounts of information than volatile memory. In some examples, the memory 124 can further be configured for long-term storage of information. In some examples, the memory 124 can include non-volatile storage elements. Examples of such non-volatile storage elements can include magnetic hard discs, optical discs, flash memories, or forms of electrically programmable memories (EPROM) or electrically erasable and programmable (EEPROM) memories.

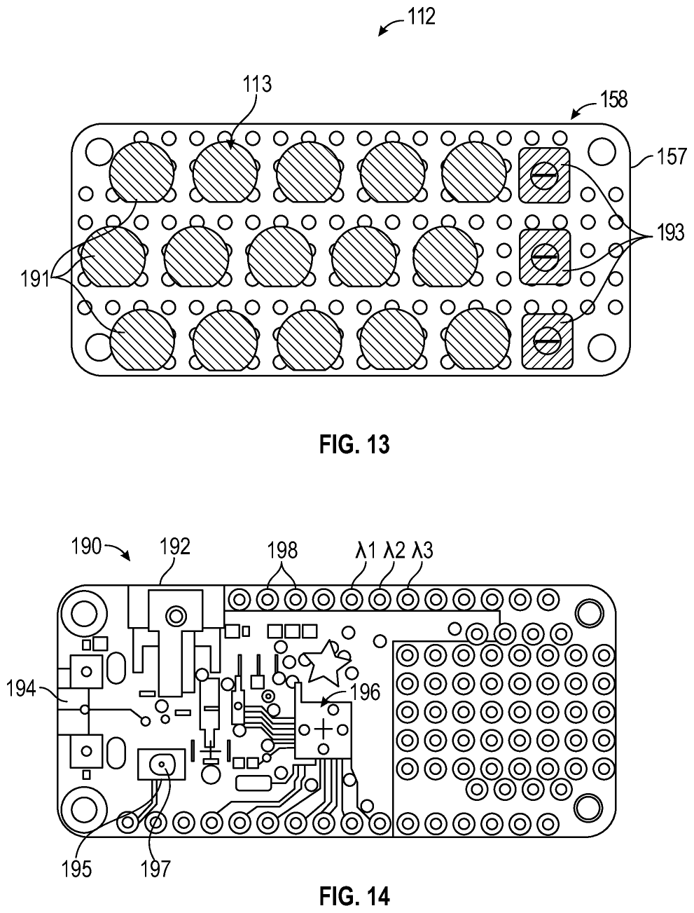

The processing circuitry 116 can thereby be capable of receiving, retrieving, and/or processing program instructions, such as stored on the memory 124 (e.g. on program memory 124P), or receiving, retrieving, and/or processing data stored on the memory 124 (e.g. on data memory 124D) to implement or otherwise execute any of, but not limited to, various functions or operations of the mobile device 102, the first camera 106, the second camera 108, or in some examples, the illumination module 112 described in this document. In still further examples, the processing circuitry 116 can be capable of receiving, retrieving, and/or processing program instructions, such stored on a memory of the second camera 108 or the processing chip 196 of the illumination module 112 (FIG. 14) to execute functions thereof.

In various examples, the memory 124 is usable by a mobile application or other software running on the processing circuitry 116 to store various program instructions for execution by the processor 122, such as to implement any of, but not limited to, the functions or operations of any of the mobile device 102, the first camera 106, the second camera 108, or the illumination module 112. For example, such a mobile application or software can be proprietarily designed or otherwise configured to implement any of documentation (e.g. image or video data collecting operations) or analysis (e.g. image, signal, or other data processing operations) of biological tissue associated with physical characteristics of a wound, injury, or other ailment of a patient. The mobile application or software can further be designed or otherwise configured to objectively monitor or track chronic physical characteristics of an injury or ailment associated the healing process.

The user interface 114 includes various input and output devices such as any of a visual display, an audible signal generator, switches, buttons, a touchscreen, a mouse, a keyboard, etc. The user interface 114 may communicate or transfer information between the imaging system 100 and a user, such as a physician. In some examples, the processor 122 can receive, retrieve and/or process instructions or data to cause the first camera 106 or the second camera 108 to activate, or repeatedly active, to collect imaging data responsive to one or more user inputs to the user interface 114 or other features, such as buttons or switches, of the mobile device 102.

In some examples, the processor 122 receives, retrieves and/or processes instructions or data to collect and/or aggregate multispectral data into the form of a three-dimensional (x, y, λ) multispectral data cube, wherein the three-dimensional multispectral data cube includes both graphical spatial dimensions of an imaged tissue area (e.g., x and y coordinates), and at least one spectral dimension (e.g., one or more spectrums of the imaged tissue area within various frequencies or wavelengths domains (λ), such as defined by the spectral ranges in which the first camera 106, the second camera 108, the optical filters 111, or the light emitters 113 are configured to capture data or otherwise operate. In some examples, the processor 122 can receive, retrieve and/or process instructions or data to analyze a three-dimensional multispectral data cube using any of a spectral decomposition algorithm (SDA), non-negative matrix factorization (NMF), independent component analysis (ICA), or principal components analysis (PCA) to analyze both spatial coordinates and reflectance or transmittance spectrums of patient tissue, such as to enable detection of physical characteristics indicative of abnormal changes that may otherwise not be obtainable from other assessment methods.

Analysis of such a three dimensional multispectral data cube can yield quantified values or ranges associated with a deep tissue injury, an extent of tissue edema, tissue oxygenation such as obtained from collecting, processing, or quantifying near infrared imaging data, quantified values or ranges associated with tissue inflammation due to infection or tissue perfusion such as obtained from infrared imaging data, or values or ranges associated with an injury or wound bioburden, or colonization estimate such as obtained from, but not limited to, a combination of near infrared and infrared imaging data.

The communications module 120 can include any of various input and output devices. The user interface 114 can utilize the communications module 120 via the processing circuitry 116 to, for example, communicate with external devices via one or more networks, such as one or more wireless or wired networks, or both. The communications module 120 can include a network interface card, such as an Ethernet card, an optical transceiver, a radio frequency transceiver, or any other type of device that can send and receive information. Other examples of such network interfaces can include Bluetooth, 3G, 4G, and Wi-Fi radio computing devices as well as Universal Serial Bus (USB) devices.

In various embodiments, the imaging system 100 includes a database 126. In some examples, the processor 122 can receive, retrieve and/or process instructions or data to send or retrieve multispectral imaging data to the database 126, such to enable various examples below. The database 126, can be, for example, an imaging database including a library, such as including a plurality of representative images, or quantified threshold values or ranges, each defining a different class or category of a similar injury or ailment. In such examples, the processor 122 can receive, retrieve and/or process instructions or data to compare a value or range of a quantified physical characteristic of a patient to the plurality of images or quantified threshold values or ranges to the classify the injury or ailment, such as to help a user assess the injury or ailment. In some examples, the database 126 can also include a plurality of historical values or ranges based on a quantified physical characteristic of biological tissue associated with an injury or ailment of an individual patient at a previous or former point in time. In such examples, the processor 122 can receive, retrieve and/or process instructions or data to compare a value or range of a quantified physical characteristic to the plurality of historical values or ranges to track a change, such as wound growth or progress, of the injury or ailment of the individual patient.

The database 126 may further contain at least one historical value or range based on a quantified physical characteristic of biological tissue associated with a similar injury or ailment of other patients collected at previous points in time. In such examples, the processor 122 can receive, retrieve and/or process instructions or data to compare a value or range of a quantified physical characteristic to the at least one historical value or range, such as to average or otherwise reference such values or ranges to improve any of the sensitivity or specificity, and thereby the accuracy of, a user assessment of a medical condition.

The power source 118 may include a battery arranged to power the imaging system 100. In other examples, the power source 118 may include an external power source, such as a charger, in electrical communication with the power source 118. In some examples, the processing circuitry 116 can be in electrical communication with an output circuit, such as realized in the form of the power source 118 and the port 103. Such an output circuit can be configured to enable transmission of electrical energy generated by the power source 118, or signal communication generated by the processing circuitry 116, to be output to the second camera 108, or in further examples, the illumination module 112.

The port 103 can include any of various signal drivers, buffers, amplifiers, or ESD protection devices, or an output terminal, such as engageable by the electrical connector 109 of the second camera 108. In further examples, the port 103 can be engageable by other components of the second camera 108, such as a battery 270 (FIG. 7) via the electrical connector 109, a charger to provide power to the power source 118, a cable connected to the power input 192 or data input 194 of the controller 188 (FIG. 14) of the illumination module 112, or a power splitter 135 (FIG. 5) to enable various combinations of the forgoing components to be in electrical communication with the mobile device 102.

In additional examples, imaging system 100 can include a wireless communication circuit, such to enable wireless electrical communication between the processing circuitry 116 and the illumination module 112. For example, a wireless communication circuit can be realized in the form of the communications module 120 located on or within the processing circuitry 116 and a communications module of a controller 188 of the illumination module 112, such as discussed with regard to FIG. 14.

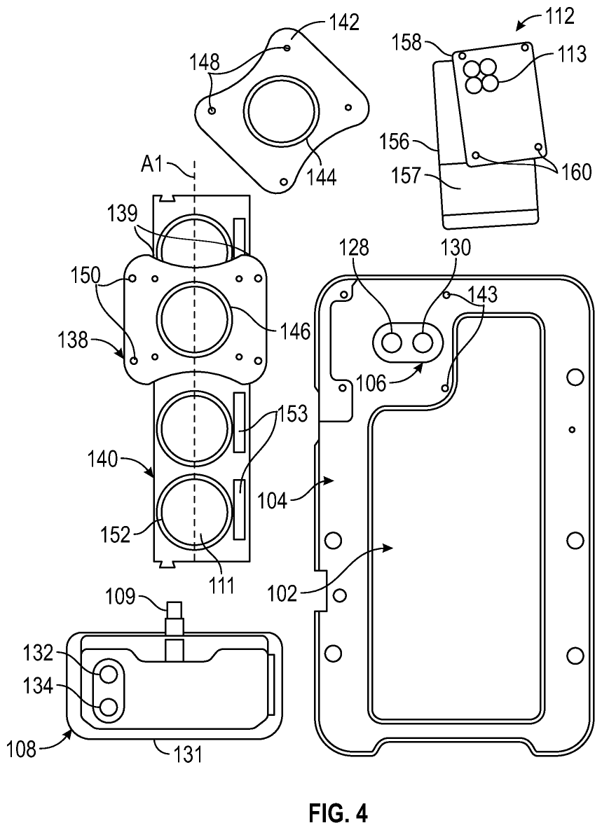

FIG. 4 illustrates a partially exploded view of an example of an imaging system 100. FIG. 5 illustrates a rear isometric view of an example imaging system 100. Also shown FIG. 5 is a first axis A1. FIGS. 4-5 are discussed with reference to the imaging system 100 discussed above in FIGS. 1-3. In some examples, the first camera 106 can be a camera integrated into the mobile device 102. The first camera 106 can include one or more cameras, or lenses, integrated into the mobile device 102, such as depending upon a make and model of the mobile device 102. For example, the first camera 106 can include a primary 128 and a second camera 130 (FIG. 4). In other examples, the primary 128 and the secondary 130 cameras can be primary 128 and secondary 130 lenses of the first camera 106.

The camera 106 can be a modified version of a CMOS camera, such as often included within various mobile devices, such as in one example of the mobile device 102. For example, the first camera 106 can be modified for near-infrared imaging by removing any near-infrared filters, such as included in many mobile device cameras. In some examples, the first camera 106 can be configured to capture images in frequencies (e.g., wavelengths) between about, but not limited to, 400 nanometers to 1600 nanometers. The infrared imaging ability of the first camera 106 can be used, for example, to observe a deep tissue injury, measure tissue edema or swelling, or measure skin oxygenation in a wound area or in healthy tissue.

In some examples, the second camera 108 can include one or more cameras, or camera lenses, integrated into a body 131 of the second camera 108, such as depending upon a make and model of the second camera 108. For example, the second camera 108 can include a primary 132 and a secondary camera 134 (FIG. 4). In some examples, the second camera 108 can be a variety of commercially available mobile thermal camera attachments, such as configured for use with various mobile devices, such as the mobile device 102. For example, the second camera 108 can be a FLIR® One camera, such as shown in FIGS. 1-2 and 4. In other examples, the second camera 108 can be a Seek® thermal camera, or still other thermal cameras designed for use with mobile devices such as smartphones. The second camera 108 can be in electrical communication with the mobile device 102 by any of various means. For example, the second camera 108 can include an electrical connector 109 insertable into the port 103 of the mobile device 102. In other examples, the second camera 108 can include wireless communication functionality, such as to communication with the communications module 120 (FIG. 3) of the mobile device 102.

The second camera 108 can be physically coupled or otherwise connected to the housing 104 using any of various means. In some examples, the electrical connector 109 can physically connect the second camera 108 to the mobile device 102, such as shown in FIGS. 1-2. In other examples, such as shown in FIGS. 5 and 7, the second camera 108 can be physically connected to the housing 104 in any of various locations using various means or methods, such as, but not limited to, fasteners such as screws or rivets, adhesives such as epoxies, tape, welding, molding, or still other means. Infrared imaging data provided by the second camera 108, such as multiple and simultaneous temperature readings of an area of biological tissue, can be used to, for example, to measure the extent of tissue inflammation from infection, or to assess tissue perfusion. In some examples, the second camera can also be configured to collect near infrared imaging data. In some examples, the second camera 108 can be configured to capture images in frequencies between about, but not limited to, 800 nanometers to 14,000 nanometers.

The filter assembly 110 can include a base 138 and a filter member 140. The base 138 can be physically coupled or otherwise connected to the housing 104 in various locations, such as generally proximal to the first camera 106, using various means such as, but not limited to, fasteners such rivets, adhesives such as epoxies, tape, welding, molding, or still other means. In an example, such as shown in FIGS. 4-5, the base 138 can be configured to interface with the housing 104 via a mount 142 positionable therebetween. In such an example, the housing 104 can define a plurality of bores 143 (FIG. 4). The bores 143 can be formed, for example, in any of various locations or orientations, such as forming a circular, square, or rectangular arrangement around the first camera 106. The bores 143 can extend transversely into the housing 104. Correspondingly, the base 138 and the mount 142 can also each define a plurality of bores 148 and 150, respectively (FIG. 4). For example, the bores 148 and 150 can be locationally or otherwise strategically formed, sized, and shaped in the mount 142 and the base 138, respectively, to align with the bores 143 when the mount 142 and the base 138 are centered, or are otherwise positioned generally proximal to, the first camera 106.

Subsequently, as shown in FIG. 5, a plurality of fasteners 149 can be inserted into and though the bores 148, and 150 to engage the bores 143 of the housing, to thereby couple the filter assembly 110 to the housing 104. The filter member 140 can generally be a body configured to receive and locate the optical filters 111 with respect each other, to the base 138. The filter member 140 can be adjustably connected to the base 138. In some examples, such as shown in FIGS. 4-5, the base 138 can be configured to receive at least a portion of the filter member 140. For example, the base 138 can define a passage 139 or other opening configured to accept and contact at least a portion of the filter member 140 in such a manner as to allow translation of the filter member 140 therethrough.

In such an example, once positioned within the base 138, the filter member 140 can be translated axially along the first axis A1 (FIG. 5) defined by the opening of the base 138 to position any of the optical filters 111 contained therein in front of, or otherwise proximal to, the first camera 106. The filter member 140 can be, for example, rectangular in shape, such as to define a plurality of apertures 152 located in a linear or otherwise in line arrangement with respect to one another. The apertures 152 can generally be openings configured to receive the optical filters 111. The apertures 152 can be configured to contact the filters 111, such as via a snap or a friction fit.

The apertures 152 can be configured to allow a user to easily remove or replace any of the optical filters 111. In some examples, the filter member 140 can define one, two, three, four, five or more separate apertures 152. The optical filters 111 can be of various shapes and sizes, for example, to conform to the dimensions of the apertures 152 defined by the filter member 140. In some examples, the optical filters 111 can be one-inch circular optical lenses. Any of the optical filters 111 can be configured for near-infrared imaging with the first camera 106. In some examples, one of the apertures 152 can be left open or blank, or otherwise left without an optical filter 111, such as to allow the camera 106 to capture traditional, or otherwise unfiltered, visible light images or video. The apertures 152 of the filter member 140 can thereby allow a user to selectively choose a wide variety of additional optical filters, such as to increase the spectral range of the imaging system 100 for a particular imaging operation.

In some examples, the filter member 140 can also include one or more labels 153. The labels 153 can correspond to, for example, the type of an optical filter 111 that is received within any of the apertures 152. For example, any of the labels 153 can specify a spectral wave or individual wavelength that each of the filters 111 may define, such to enable a user to easily position one of the optical filters 111 in front of the camera 106. In some examples, the filter member 140 can form other shapes, such as a radial or circular shape, such rotatable relative to the base 138 to position any of the optical filters 111 contained therein in front of, or otherwise proximal to, the first camera 106. In some examples, the base 138 can also include a spring detent engageable with the filter member 140, such as to help prevent unintended movement of the filter member 140, and thereby the optical filters 111, relative to the first camera 106.

In still further examples, the filter member 140 can be mounted on an automatic, such as a timed, wheel or other mechanical mechanism configured to translate or rotate the optical filters 111 in front of the first camera 106. In one such example, the filter assembly 110 can include a mechanically timed mechanism, such as configured to be synchronized with the illumination module 112, to sequentially position at least two of the optical filters 111 proximally, or in front of, the first camera 106, such as during emission of light in at least two different wavelengths emitted by the illumination module 112. In some examples, the imaging system 100 can include a power splitter 135. The power splitter 135 can be a dual power splitter, a triple power splitter, or other power splitters. The power splitter 135 can be configured to allow various components of the imaging system 100, such as the second camera 108 or the illumination module 112, to concurrently receive power from the power source 118 (FIG. 3) of the mobile device 102 via the port 103 (FIG. 2). For example, the power splitter 135 can concurrently engage the port 103 and an electrical connector (e.g., the electrical connector 109) of the second camera 108 or an electrical connector associated with the illumination module 112.

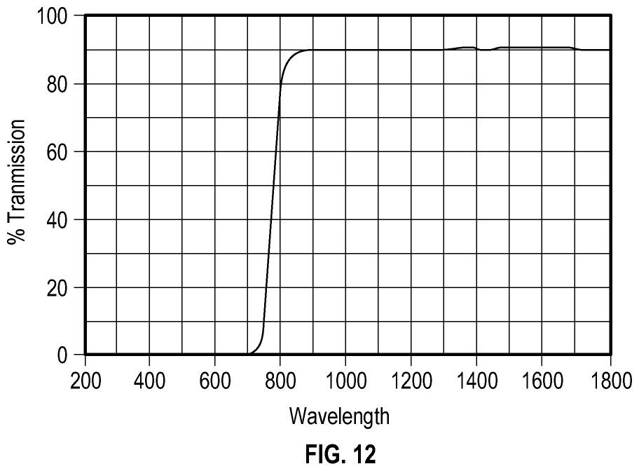

In some examples, the illumination module 112 can include, in addition to the light emitters 113, a casing 156, a power supply 157, and a circuit board 158 (FIG. 4). The casing 156 can be a housing, such as configured to partially, or completely, encompass the power supply 157 and the circuit board 158, or a mount, such as configured to be positioned between the circuit board 158 and the housing 104. The casing 156 can be configured, for example, to interface directly with the housing 104 of the mobile device 102, such as to couple the illuminator module 112 to the housing 104 in any of various locations with respect to the housing 104. The casing 156 can be physically coupled or otherwise connected to the housing 104 using any of various means or methods such as, but not limited to, fasteners such rivets, adhesives such as epoxies, tape, welding, molding, or still other means. The circuit board 158 can be a custom or commercially available printed or customizable, such as a development, circuit board. The circuit board 158 is shown in detail in FIGS. 13-14 below.

The circuit board 158 can include the light emitters 113. In some examples, the light emitters 113 can be powered via a physical connection from the circuit board 158 to the power supply 157. The power supply 157 can be a battery, such as positionable within the casing 156. The power supply 157 can allow the illumination module 112 to have a self-contained power supply separate from other components of the imaging system 100. In other examples, the light 113 emitters can be powered directly from the power source 118 (FIG. 3) of the mobile device 102, such as via cable extending between the port 103 of the mobile device 102 to the circuit board 158. The circuit board 158 can be physically coupled, or otherwise connected, to any of the casing 156, the power supply 157, or the controller 188 shown in FIG. 13, via connecting features 160. The connecting features 160 can be any of, but not limited to, fasteners such rivets, adhesives such as epoxies, tape, welds, a molding, or still other fastening or connecting means.

The illumination module 112 may further include user inputs devices such as a plurality of buttons 162 or a switch 164 coupled to the circuit board 158. The buttons 162 and the switch 164 can be configured to enable a user to configure variations operations of the illumination module, such as including any of a wavelength of light to be emitted by the light emitters 113, a number of different wavelengths of light to be emitted by the light emitters 113, a cycle length of the illumination module 112 defined by a time interval or period between activation and deactivation of the light emitters 113, or a cycle quantity of the illumination module 112, such as a number of cycles (e.g., activations and deactivations of the light emitters 113) that the illumination module 112 is configured to perform without any additional user inputs to, for example, the buttons 162 or the switch 164 upon activation or otherwise first emitting light responsive to a user input.

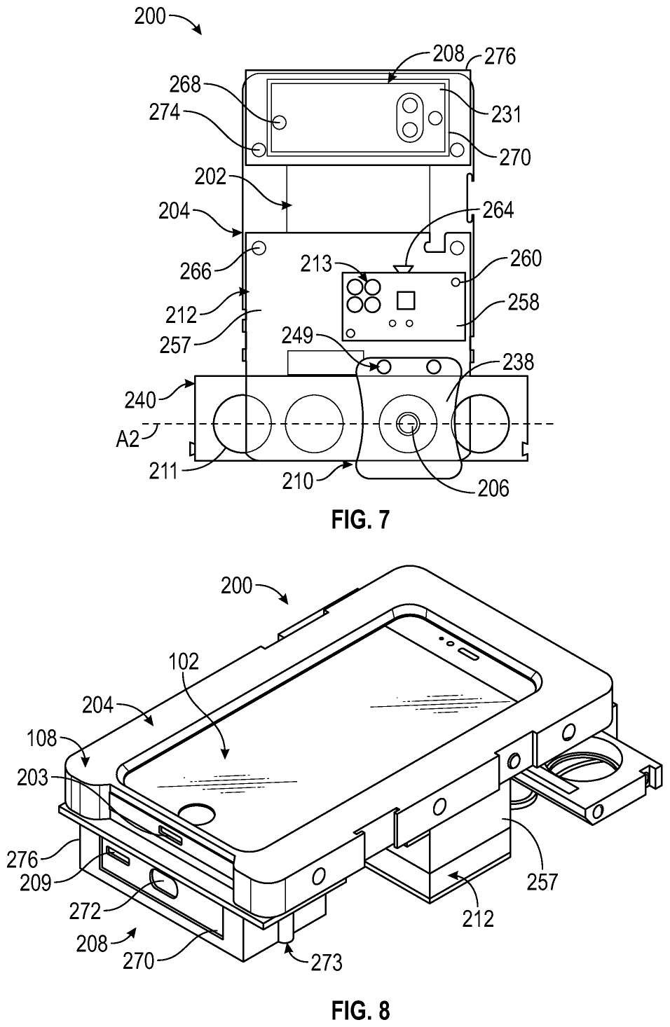

FIGS. 6-8 illustrate front, rear, and side views, respectively, of an example of an imaging system 200. FIGS. 6-8 are discussed below concurrently. The imaging system 200 can include any of the components of the imaging system 100 shown in, and discussed with reference to, FIGS. 1-5 above and the imaging system 100 discussed above can be modified to include the components of the imaging system 200. FIGS. 6-7 also show a second axis A2. The imaging system 200 can include a mobile device 202, a housing 204, a first camera 206, a second camera 208, a filter assembly 210, and an illumination module 212, each of which, including any component thereof, can be similar or different to the mobile device 102, the housing 104, the first camera 106, the second camera 108, the filter assembly 110, or the illumination module 112, respectively, of the imaging system 100.

The filter assembly 210 and the illumination module 212 can be physically coupled or otherwise connected to the housing 204 in a different orientation or position relative to, for example, the filter assembly 110 and the illumination module 112 of the imaging system 100 shown in FIGS. 1-2 and 4-5. In such an example, the housing 204, and any feature thereof, can be configured differently to the housing 104 shown in FIGS. 1-2 and 3-5, such as based on the orientation or position of any of the second camera 208, the filter assembly 210, or the illumination module 212 on, or relative to, the housing 204. In some examples, the filter assembly 210 can be coupled to the housing 204 in an orthogonal orientation relative to the orientation of the filter assembly 110 shown in FIG. 5. In such an example, once a filter member 240 of the filter assembly 210 is positioned within a base 238 of the filter assembly 210, the filter member 240 can be translated axially along the second axis A2 (FIGS. 6-7) defined by the passage 139 (shown in FIG. 5) of the base 238 to position any of the optical filters 211 (FIG. 7) contained therein in front of, or otherwise proximal to, the first camera 206 (FIG. 7).

In some examples, the illumination module 212 can include a power supply 257. In some examples, such as shown in FIGS. 7-8, the power supply 257 of the illumination module 212 can extend generally perpendicularly to the power supply 157 shown in FIG. 5. For example, the power supply 257 can extend substantially or completely across the housing 204, as shown in FIG. 7. In one example, the power supply 257 can be a 1,200mAh lithium-polymer battery. Other battery types and specifications can be used without departing from the teachings of the present subject matter. In such examples, the power supply 257 can be coupled to the housing 204 via connecting features 266. The connecting features 266 can be any of, but not limited to, fasteners such rivets, adhesives such as epoxies, tape, welds, a molding, or still other fastening or connecting means. In such examples, a circuit board 258 of the illumination module 212, such as including a switch 264, can be coupled to the power supply 257 or to the housing 204 via the connecting features 260. The connecting features 260 can be any of, but not limited to, fasteners such rivets, adhesives such as epoxies, tape, welds, a molding, or still other fastening or connecting means.

In some examples, the imaging system can include a battery 270 (FIGS. 7-8). The battery 270 can allow the second camera 208 to have a self-contained power supply separate from other components of the imaging system 100. In some examples, the imaging system 100 can include a battery box 276 (FIGS. 7-8). In such examples, the battery box 276 can be configured to partially, or completely, encompass the battery 270. The battery box 276 can be physically coupled, or otherwise connected to, the housing 204 via connecting features 274 (FIG. 7), such as to support the battery 270 with respect to the housing 204. The connecting features 274 can be any of, but not limited to, fasteners such rivets, adhesives such as epoxies, tape, welds, a molding, or still other fastening or connecting means. For example, the connecting features 274 can be including in, or can otherwise extend transversely through, bores 273 (FIG. 8) defined by the battery box 276 to engage the housing 204.

In some examples, the second camera 208 can be physically coupled or otherwise connected to the housing 204, or to the battery box 276, via connecting features 268 (FIG. 7). The connecting features 268 can be any of, but not limited to, fasteners such rivets, adhesives such as epoxies, tape, welds, a molding, or still other fastening or connecting means. In such examples, the electrical connector 209 shown in FIG. 8 can be a port, such as configured to receive one end of a cable in electrical communication with the battery 270. For example, the battery 270 can include a charging port 272. In such an example, a cable can concurrently engage the electrical connector 209 and the charging port 272 of the battery 270, such as to enable the second camera 208 to receive power from the battery 270. The charging port 272 can also be configured to receive a charge via engagement with an electrical connector, such as connected to an external charger, or connected to the port 103 (FIG. 2), or the power splitter 135 (FIG. 5), to receive a charge from the power source 118 (FIG. 3) of the mobile device 102. In additional examples, the second camera 208 can be powered directly from the power source 118 (FIG. 3) of the mobile device 102, such as via a cable extending between the port 103 (FIG. 2), or the power splitter 135 (FIG. 5) to the electrical connector 209 (FIG. 8) of the second camera 208.

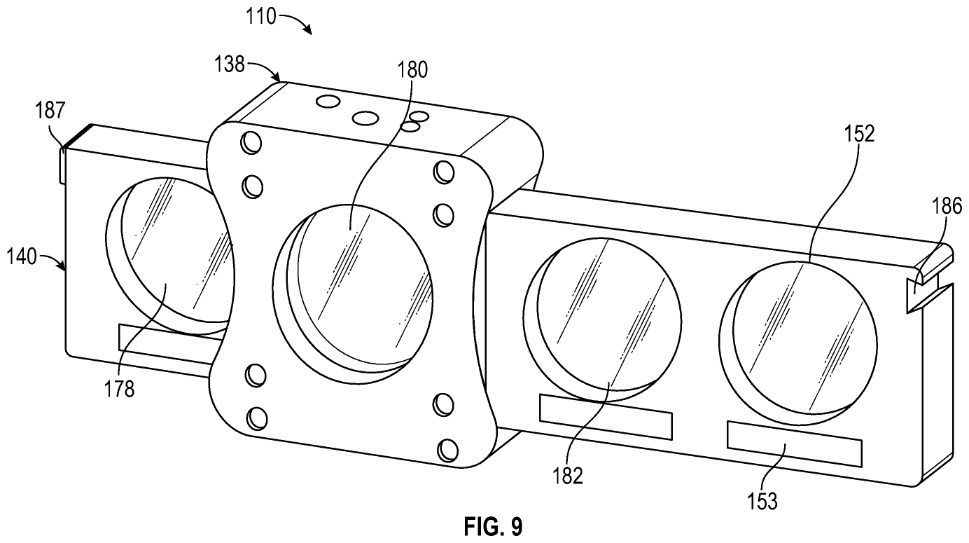

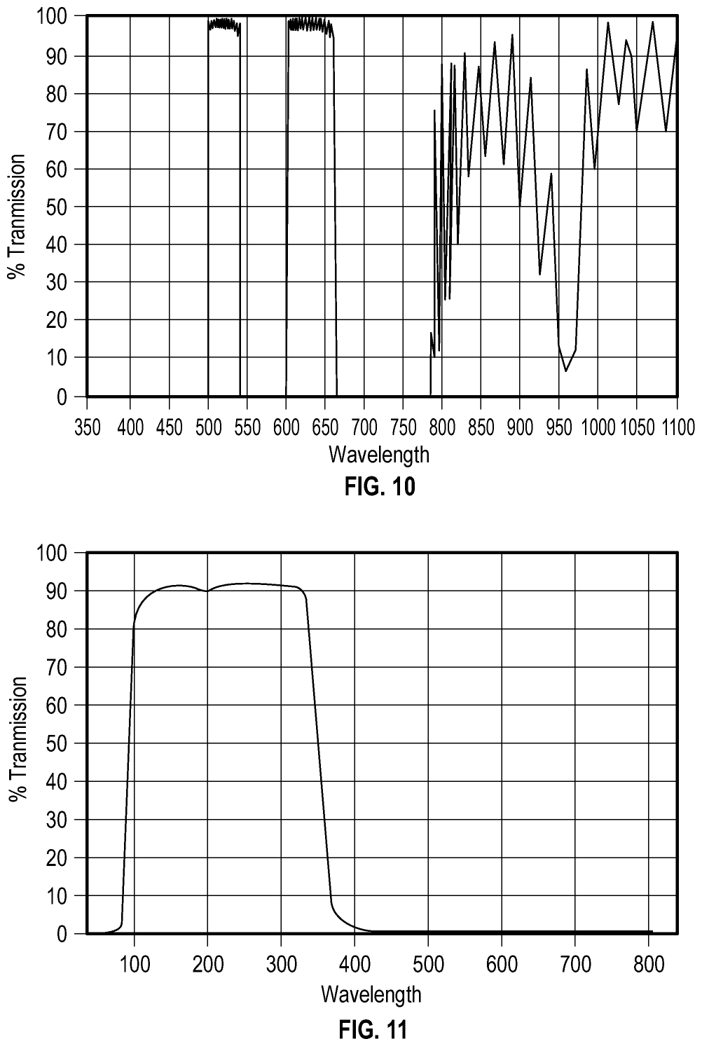

FIG. 9 illustrates a front isometric view of an example filter assembly 110. FIGS. 10-12 illustrate spectral graphs of various optical filters of an example filter assembly 110. FIG. 9-12 are discussed below concurrently. FIGS. 9-12 are discussed with reference to the imaging system 100 or the imaging system 200 shown above. In some examples, such as shown in FIG. 9, the filter assembly 110 can be a commercially available filter module, such as a Thorlabs® opto-mechanical filter module. The filter assembly 110 can allow a user to change or otherwise broaden the spectral imaging range of the imaging system 100 by any of various methods. For example, a user can translate the filter member 140 to position any of the optical filters 178, 180, 182, or 184 shown in FIG. 9 in a position proximal to the first camera 106, such as to choose the spectral range in which the first camera 106 can collect imaging data, such as images or videos. The optical filters 178, 180, 182, or 184 can be similar or different to the optical filters 111 shown in, for example, but not limited to, FIGS. 4-5.

In some examples, a user can easily remove or replace any of the optical filters 178, 180, 182, or 184 such, such as by inserting or removing any of the optical filters 178, 180, 182, or 184 into or from the apertures 152 (FIG. 4), such as to further broaden the spectral range in which multispectral imaging can be performed using the imaging system 100 (FIG. 1-5) or 200 (FIGS. 6-8). In still further examples, the filter member 140 can include a first expansion feature 186 or a second expansion feature 187. For example, the first expansion feature 186 can be a protrusion and the second expansion feature 187 can be recess, such as configured to engage similar features on other filter members. In one such example, a second filter member can be coupled to the filter member 140 to expand the number of apertures 152 from four, such as shown in FIG. 9, to eight apertures.

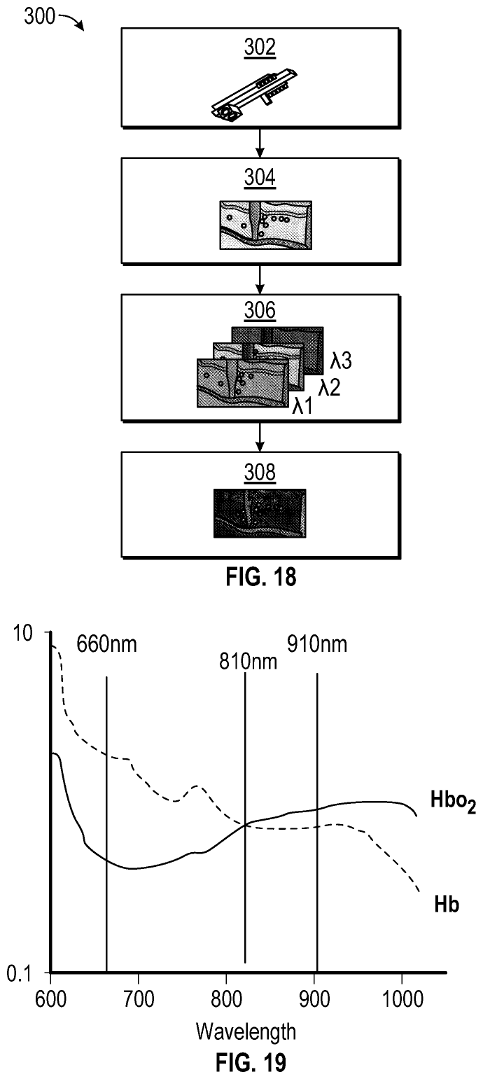

Any of the features or operations of the filter assembly 110 discussed above can be beneficial for a user, as different biochemical constituents or biochemical changes of a patient can have different spectral signatures between certain wavelengths or frequencies. For example, a user can utilize and of the above features to configure the filter assembly 110 to select a particular spectral range for imaging biological tissue of a patient, such as based on a particular injury, wound, or ailment of the patient associated with the biological tissue to be imaged. In some examples, a spectral range of about, but not limited to, 500 to 800 nanometers can be used to help map the oxygen saturation of biological tissue, such as to cover the spectral signatures of HbO2 and Hb (exemplified in FIG. 19). In some examples, such as for multispectral fluorescence imaging, a spectral range of about, but not limited to, 400 to 750 nanometers can be used to observe the excitation spectral signatures of many fluorescent probes or dyes, which are typically located in this wavelength region. In some examples, a spectral range of about, but not limited to, 900 to 1700 nanometers can be used for a tooth disease diagnosis, as dental enamel can manifest a high transparency for near-infrared light.

In some examples, any of the optical filters 178, 180, 182, or 184 can include one of a spectral filter such as a near infrared longpass filter, a bandpass filter, a dual-bandpass filter, a polarization filter, a visible filter, an ultraviolet filter, or a reference or blank filter, such as to function as a reference point. In some examples, the optical filters 178, 180, 182, and 184 can be commercially available optical filters. For example, any one of the optical filters 178, 180, 182, or 184 can be a Chroma dual bandpass filter, such as shown in FIG. 10. In some examples, one of the optical filters 178, 180, 182, or 184 can be a Midwest Optical® Visible bandpass filter, such as shown in FIG. 11. In some examples, one of the optical filters 178, 180, 182, or 184 can be a Thorlabs® 780 nm longpass filter, such as shown in FIG. 12. In such examples, the Midwest Optical Visible filter can prevent near-infrared light energy from leaking into the Chroma dual band pass filter of green and red. In any of FIGS. 10-12, the X (e.g. horizontal), and Y (e.g., vertical) axes can represent wavelength, in nanometers, and percentage of transmittance or transmission, respectively.

In some examples, one of the apertures 152 of the filter assembly can be left empty to allow light to pass through the apertures 152 un-attenuated, such for usage for infrared imaging or in still further examples, use with various examples of the second camera 108, such as in an example where the second camera 108 is configured to also capture imaging data in visible or near infrared light spectrums. In some examples, at least three of the optical filters 178, 180, 182, or 184 can be configured for use during emission of at least three wavelengths of light that the illumination module 112 is configured to emit, such as discussed in further detail in FIGS. 13-14 below.

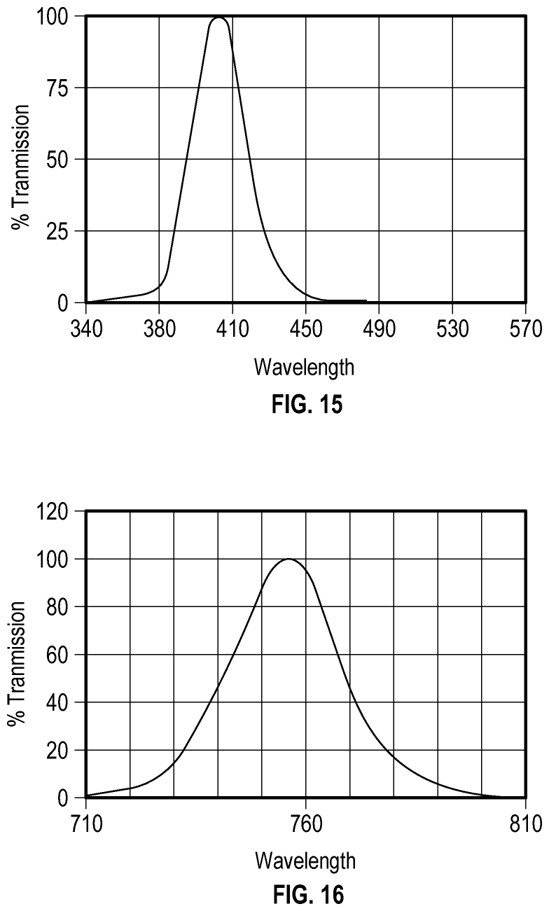

FIGS. 13 illustrates a front isometric view of an example illumination module 112. FIG. 14 illustrates a front isometric view of a controller 188 of an example illumination module 112. FIGS. 13-15 are discussed with reference to the imaging system 100 or the imaging system 200 shown above. FIGS. 15-17 illustrate spectral graphs of various wavelengths of light emission of an example illumination module 112. FIGS. 13-14 are discussed below concurrently. The illumination module 112 can be realized as a combination of the circuit board 158 (FIGS. 4-5 and 13) and a controller 188 (FIG. 14).

The circuit board 158, as discussed above, can generally be a printed circuit board including various numbers or combinations of the light emitters 113. In some examples, the light emitters 113 can be light emitting diodes (LEDs) epoxied, soldered, or otherwise physically and electrically coupled to the circuit board 158. In some examples, the light emitters 113 can be configured to define two, three, four, five, six, or still other numbers of groups. The light emitters 113 can be separately or sequentially activatable to emit light of different wavelengths, relative to one another. In some examples, such as shown in FIG. 13, the light emitters 113 can be configured to define groups 191. In some examples, each of the group 191 can include five light emitters 113 configured to activate concurrently to emit light in one particular or specific wavelength. In some examples, one of the groups 191 of light emitters 113 can be configured to be activatable to emit light in 405 nanometers (FIG. 14), one of the group of light emitters 191 can be configured to emit light in 760 nanometers (FIG. 15), and one of the groups 191 of light emitters can be configured to emit light in 850 nanometers (FIG. 16), respectively. However, any number of other wavelengths are possible without departing from the scope of the present subject matter. In some examples, each of the light emitters 113 can define various dimensions, such as, but not limited to, 2-4 millimeters, 5-7 millimeters, or 8-10 millimeters in diameter.

In any such examples, the groups 191 of the light emitters 113 can be independently biased, such as via separate bias resistors 193. In some examples, the circuit board 158 can include three separate bias resistors 193, such as each corresponding to one of the groups 191 of the light emitters 113. The separate bias resistors 193 can be epoxied, soldered, or otherwise physically and electrically coupled to the circuit board 158.

In some examples, the illumination module 112 can also include a diffuser screen, such as generally configured as a grid to individually separate each of the light emitters 113, or each of the groups 191. Such a diffuser screen can individually separate one wavelength of light emitted by any of the light emitters 113 from another wavelength of light emitted by any of other light emitters. In some examples, such a diffuser screen can help to smooth out illumination patterns projected by the groups 191 onto a biological tissue of a patient associated with a wound, injury, or ailment of the patient. Alternatively, each of the groups 191 can be fitted with an individual diffuser screen, such as to compensate for wavelength dependent transmission to achieve a uniform intensity output pattern. In any of FIGS. 10-12, the X (e.g. horizontal), and Y (e.g., vertical) axes can represent wavelength, in nanometers, and percentage of transmittance or transmission, respectively.

Regarding FIG. 14, the controller 188 can be a microcontroller, such as realized in the form of a variety of commercially available or custom printed circuit boards. In some examples, such as shown in FIG. 14, the controller 188 can be an Adafruit® Feather M0 Basic Proto, or other SMART-ARM based microcontroller from Adafruit®. The controller 188 can be configured to interface with the circuit board 158 (FIG. 13), such that the controller 188 is in electrical communication with the circuit board 158. The controller 188 can include a variety of GPIO pins, USB-to-serial program, and built in debug capabilities, without need for an FTDI-like chip.

The controller 188 can include a power input 192, a data input 194, and a processing chip 196. The power input 192 can be configured to receive electrical power from, for example, an external power source such as a battery. The controller 188 can include built-in charging capability for a battery, such as for a 3.7V lithium-polymer battery. In some examples, the power input 192 can receive power directly from the power source 118 (FIG. 3) of the mobile device 102, such as via a cable extending between the port 103 (FIG. 2), or the power splitter 135 (FIG. 5), and the power input 192. The processing chip 196 can include a processor and memory configured to store the instructions. Operation of the processing chip 196 can be similar to the processor 122 discussed in FIG. 3, at least in that the processing chip 196 can be capable of receiving, retrieving, and/or processing program instructions, such as stored internal memory of the processing chip 196, to implement or otherwise execute any of, but not limited to, various functions or operations of the illumination module 112 described in this document.

The data input 194 can be, for example, a Micro-USB jack for power and/or USB uploading. For example, a user can add, change, or otherwise configure program instructions stored on internal memory of the processing chip using, for example, any of various computers systems or programming devices in communication with the data input 194. Such programming instructions can be easily changed by a user to accommodate specific multispectral imaging requirements. For example, the controller 188 can be configured to receive a user-input to control or otherwise configure such programming instructions. In some examples, such a user input can be actuation or other inputs to the buttons 162 or the switch 164, such as discussed in FIG. 5. In such an example, the controller 188 can be configured to enable functionality of the buttons 162 or the switch 164.

In some examples, such a user input can be a user input to the user interface 114 (FIG. 1) of the mobile device 102. In such an example, the controller 188 can be in electrical communication with the mobile device 102 via the data input 194, such as to receive data or program instructions directly from the processing circuitry 116 (FIG. 3) of the mobile device 102. In one such example, electrical communication can be established between the controller 188 and the mobile device 102 via a cable extending between the port 103 (FIG. 2), or the power splitter 135 (FIG. 5), and the data input 194. In other such examples, the controller 188 can be coupled to, or otherwise include, a communication module to wirelessly receive data or program instructions directly from the communications module 120 (FIG. 3) of the mobile device 102.

In such examples, the controller 188 can include a network interface card, such as an Ethernet card, an optical transceiver, a radio frequency transceiver, or any other type of device that can send and receive information. Other examples of such network interfaces can include Bluetooth, 3G, 4G, and Wi-Fi radio computing devices as well as Universal Serial Bus (USB) devices. In still further examples, the communications module 120 (FIG. 3) of the mobile device 102 can include a radio frequency identification (RFID) or near-field communication (NFC) wireless transceiver, and the controller 188 can include, or be coupled to, an RFID or NFC passive or active tag. In some examples, the controller 188 can further include, or be coupled to, various other input and output devices such as any of a visual display, an audible signal generator, switches, buttons, a touchscreen, a mouse, a keyboard, etc., such as to receive data or program instructions or display such program instructions to a user.

In view of the above, the controller 188 can be configured to control the illumination module 112, such as by controlling various aspects of activation and deactivation of the light emitters 113 (FIG. 13), including activation and deactivation of the groups 191 (FIG. 13) relative to one another. In some examples, the controller 188 can enable a user to configure a wavelength of light to be emitted by the light emitters 113, a number of different wavelengths of light to be emitted by the light emitters 113, a cycle length of the illumination module 112 defined by a time interval or period between activation and deactivation of the light emitters 113, or a cycle quantity of the illumination module 112, such as a number of cycles (e.g., activations and deactivations of the light emitters 113) that the illumination module 113 is configured to perform.

In one example, controller 188 can be configured to automatically cycle the groups 191 through various cycle quantities, such that each of the groups 191 emits light in one wavelength for about, but not limited to, five seconds, and each of the groups 191 emits light in a different wavelengths relative to one another. In some examples, the light emitters 113 can be in electrical communication with the controller 188, and thereby the processing chip 196, via one or more of a plurality of input/outputs 198. In one example, three of the plurality of input/outputs 198 can be configured to correspond to and control each of the groups 191 of the light emitters 113 shown in FIG. 13. In such an example, each of the groups 191 can individually activated by the controller 188 to emit light in a one specific wavelength (e.g., λ1, λ2, and λ3 as shown in FIG. 14). The controller 188 can thereby sequentially activate the groups 191 to cause the illumination module 112 to emit light in three different wavelengths. In other examples, more or less of the input/outputs 198 can be configured to correspond to and control more or less of the light emitters 113.

In some examples, the controller 188 can include a shut off control 195 to control the cycle time of the illumination module 112. The shut off control 195 can be implemented in hardware, such as a stop button 197, or in software, such as the program instructions are described above. The shut off control 195 can be auto-timed, such as pre-set via a user input using any of, but not limited to, the buttons 162, switch 164, or user interface 114 (FIG. 1), to control the cycle length or the cycle quantity of the illumination module 112. In one example, the shut off control 195 can be configured to automatically stop activation of the light emitters 113 (FIG. 13) after about, but not limited to, thirty seconds after activation, or after about thirty seconds of continuous or intermittent activation and deactivation.

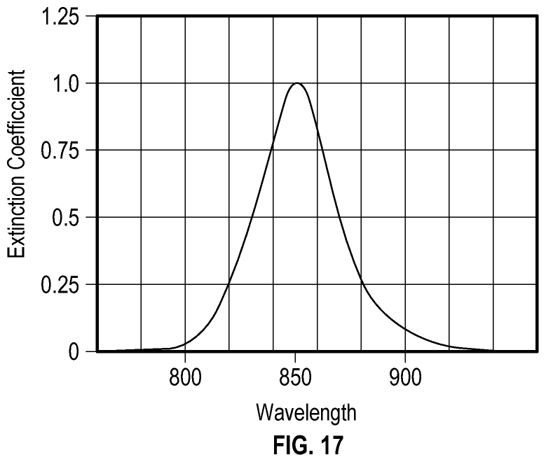

FIG. 18 illustrates a method 300 of assessing an injury or ailment of a patient using a mobile imaging system. FIG. 19 illustrates a graph comparing Oxyhemoglobin (HbO2) and deoxyhemoglobin (Hb) Isosbestic characteristics. FIGS. 18-19 are discussed with reference to the imaging system 100 or the imaging system 200 shown above. FIGS. 18-19 are discussed below concurrently.

The method 300 can include operation 302. The operation 302 can include configuring the mobile imaging system based on the injury or ailment including configuring processing circuitry of a mobile phone arranged to control a near-infrared camera and an infrared camera. For example, a user can obtain and activate the mobile imaging system, such as using a user interface of, or other input features such as buttons or switches, of a mobile device, such as a mobile phone. In some examples, a user can configure various operations of the mobile imaging system, such as via one or more user inputs to various components of the mobile imaging system. For example, a user can configure processing circuitry of the mobile device using a user interface of the mobile device, such as to control a camera system operable to capture visible, near infrared, or infrared images or video. In some examples, a user can configure a filter assembly of the mobile imaging system, such as by inserting or replacing various optical filters received within a filter member, or by translating the filter member to position any of the optical filters received therein in front of a camera.

In some examples, a user can configure an illumination module of the mobile imaging system, such as via one or more user inputs to input features thereof, such as buttons or switches, or other devices configured or otherwise operable to configure program instructions of the illumination module, such as to control a wavelength of light to be emitted, a number of different wavelengths of light to be emitted, a cycle length of the illumination module, or a cycle quantity of the illumination module. In some examples, the operation 302 can first comprise introducing a fluorescent dye into internal anatomy of the patient. In such examples, a user can configure the filter assembly by positioning an optical configured for multispectral fluorescence imaging in front of a camera.

The method 300 can include operation 304. The operation 304 can include collecting multispectral imaging data of biological tissue associated with the injury or ailment, wherein the multispectral imaging data includes at least a plurality of near-infrared and infrared images. For example, a user can operate the mobile imaging system, such as via one or more user inputs to the user interface of the mobile device, to cause a camera system to capture images or video in a combination of visible, near infrared, or infrared light spectrums. In some examples, the operation 304 can include sequentially positioning at least two optical filters in a position proximal to the near-infrared camera. For example, a user can translate a filter member of a filter assembly to position any of a plurality of optical filters received therein in front of a camera of the mobile imaging system configured to capture near infrared images.

In some examples, the operation 304 can include sequentially illuminating the biological tissue with at least two wavelengths of light. For example, a user can operate an illumination module of the mobile imaging system, such as via one or more user inputs to input features thereof, such as buttons or switches, or devices configured to configure program instructions of the illumination module, to cause the illumination module to sequentially activate at least two groups of light emitters configured to emit light in different wavelengths relative to each other. In some examples, the operation 304 can include collecting a three-dimensional multispectral data cube. For example, a user can configure processing circuitry of a mobile device to collect or aggregate multispectral imaging data into the form of a three-dimensional (x, y, λ) multispectral data cube. For example, the three-dimensional multispectral data cube can be visualized as a three dimensional cube including a face, such as defined by a function of spatial coordinates (x, y) from a plurality of collected two-dimensional multispectral images, and a depth, such as defined by a function of a spectral dimension (λ) from the wavelength or spectral range in which the multispectral images were captured within.

In some examples, the plurality of multispectral images can include images or video captured in two, three, four, five, or still other numbers of spectral rages. In some examples, the plurality of multispectral images can include images or video captured in three wavelengths (e.g., λ1, λ2, or λ3) as shown in FIG. 18. In some examples, any of the multispectral imaging data collected can be multispectral fluorescence imaging data, such as of biological tissue including or associated with a fluorescent marker or dye deposited thereon or therein. In some examples, operation 304 can be performed or otherwise implemented indoors to reduce ambient light, such as to help avoid noise and improve a Signal-to-Noise ratio during collection of the multispectral imaging data.

The method 300 can include operation 306. The operation 306 can include quantifying a physical characteristic of the biological tissue associated with the injury or ailment using the processing circuitry, wherein the physical characteristic includes at least one of tissue edema or swelling, tissue oxygenation, tissue perfusion, bacterial load, bioburden, a wound area, or a wound volume. For example, a user can configure processing circuitry of a mobile device, such as a mobile phone, to analyze a three-dimensional multispectral data cube, such as via a mobile application or other software running on the processing circuitry. For example, the processing circuitry of the mobile device can perform any of a spectral decomposition algorithm (SDA), non-negative matrix factorization (NMF), independent component analysis (ICA), or principal components analysis (PCA) to analyze both spatial coordinates and reflectance or transmittance spectrums the biological tissue, such as to enable detection of physical characteristics indicative of abnormal changes that may otherwise not be obtainable from other assessment methods.

The operation 306 can also include a variety of types of image processing. For example, a user can configure processing circuitry of a mobile device, such as a mobile phone, to perform or otherwise implement image processing such as including averaging conducted over a large number of physiologically relevant pixels to help improve the definition of a resulting photoplethysmogram (PPG) signal. A PPG signal can include a pulsatile (AC) component, such as provided by cardiac synchronous variations in blood volume from heartbeats, and a superimposed DC component shaped or otherwise defined by respiration, sympathetic nervous system activity, or thermoregulation.

In some examples, image processing can include reducing sensor noise amplitude by a factor, such as equal to the square root of a number of pixels used during averaging, to estimate oxygenation and heart rate data. In such an example, a user can select an image area as region of interest, such as to be quantified or otherwise analyzed, wherein the region of interest is a specific wavelength (λ). In some examples, the region of interest can be an area of biological tissue associated with a wound, injury, or ailment of patient (such as m×n pixels, or a 1024×1280 image). In other examples, other sizes of images, or portions of images, can be used or selected as a region of interest. In some examples, the region of interest can be a preset or predetermined region of interest programmed or otherwise entered into the mobile device, or into any of a first or a second camera of the mobile imaging system, such as using a mobile application or other software running on processing circuitry of the mobile device.

In some examples, a region of interest can be manually chosen or otherwise selected by a user after imaging processing. In some examples, when a region of interest is being analyzed, an AC/DC normalization step or process can be performed or otherwise implemented by processing circuitry of the mobile device, such as to help prevent a small change in the distance or positioning of the imaging system 100, relative to a patient, from affecting the multispectral data collected in operation 304. Further, baseline patient oxygenation measurements, such as collected using a commercially available contact pulse oximeter, can also be recorded concurrently with operations of the mobile imaging system, such as to help improve the accuracy of estimated tissue oxygenation values or ranges quantified by the mobile imaging system. For example, tissue oxygenation values or ranges collected using the pulse oximeter can be used as a reference, or otherwise compared, against values or ranges obtained using the mobile imaging system. In some examples, the operation 306 can include keeping a patient being imaged with the mobile imaging system as still as possible, such as to help reduce moving or other image artifacts that can affect signal processing, or other aspects, of multispectral data analysis.

In some examples, the operation 306 can also include a variety of types of signal processing. For example, a user can configure processing circuitry of a mobile device, such as mobile phone, to perform or otherwise implement signal processing including any of: (1) obtain a region of interest for each wavelength, such as an area of 150×150 pixels, (2) average the region of interest intensity signal to obtain PPG signals at corresponding wavelength, (3) AC/DC normalization, (5) fast Fourier transform (FFT), (6) find local maximum, (7) extract heart rate peak frequency, (8) calculate relative risk (RR), (9), extract SpO2, (10) display signal or color map to a user.

In some examples, the plurality of images collected by the mobile imaging system can be quantified or otherwise analyzed in real-time, such as during collection, or at a later time, such as after being stored on a memory of the processing circuitry of the mobile device, or on a remote database. In any of various examples, multispectral imaging data collected by the mobile imaging system can be a combination of any of images or video captured using spectral ranges such as, but not limited to, a visible light spectrum, such as between about, but not limited to, 0.4 to 0.7 micrometers, a near infrared light spectrum, such as between about but not limited to, 0.7 to 1 micrometers, a short-wave infrared light spectrum, such as between about but not limited to, 1 to 1.7 micrometers, a mid-wave infrared light spectrum such as between about, but not limited to, 3.5 to 5 micrometers, or a long-wave infrared light spectrum, such as between about but not limited to, 8 to 12 micrometers in wavelengths. Data in such spectral ranges can be collected using, for example, any of a first camera, second camera, a filter assembly, or an illumination module of the mobile imaging system.

In any of the above examples, the operation 306 can yield, but is not limited to, quantified values or ranges associated with a deep tissue injury, an extent of tissue edema, tissue oxygenation such as obtained from collecting, processing, or quantifying near infrared imaging data, quantified values or ranges associated with tissue inflammation due to infection or tissue perfusion such as obtained from infrared imaging data, or values or ranges associated with an injury or wound bioburden, or colonization estimate such as obtained from a combination of near infrared and infrared imaging data.

In some examples, tissue oxygenation values or ranges yielded during operation 306 can provide information about a patient's peripheral circulation, such as to help assess various medical conditions of the patient. Regarding tissue oxygenation values or ranges yielded during operation 306, a mobile application or software running on the processing circuitry of the mobile device can include, perform, or otherwise implement, for example, an algorithm configured to measure photoplethysmographic (PPG) signals at two or more different wavelengths (λ). Such an operation is often conducted by a commercially available pulse oximeter to obtain an estimate oxygen saturation (SpO2) value or range from one contact site of a patient's body. Such an algorithm can also, for example, compare Isosbestic characteristics of Oxyhemoglobin (HbO2) and Deoxyhemoglobin (Hb) at, such as, but not limited to, 810 nanometers (as shown in FIG. 19) as a reference to correct for image artifacts such as shadows, reflections, scattering, and other variations in pixel-to-pixel sensitivity of an imaging sensor, such as realized as combination of any of a mobile device, a first camera, a second camera, a filter assembly, or an illumination module of the mobile imaging system. Various image artifacts can be a problem associated with integrated smartphone cameras, such as, for example, a first camera of the mobile imaging system.

The method 300 can include operation 308. The operation 308 can include classifying the injury or ailment by comparing, using the processing circuitry, a value or range of the quantified physical characteristic to a library of threshold values or ranges, each defining a different class or category of the injury or ailment. The operation 308 can be based on data analyzed or quantified during the operation 306. The operation 308 can include classifying the injury or ailment by comparing, using the processing circuitry, a value or range of the quantified physical characteristic to a library of threshold values or ranges, each defining a different class or category of the injury or ailment. For example, the mobile device can be in communication with an imaging database including a library, such as including a plurality of representative images, or quantified threshold values or ranges, each defining a different class or category of a similar injury or ailment.

In such an example, a user can operate the mobile imaging system, such as via one or more user inputs to the user interface of the mobile device, to cause the processing circuity to the classify the injury or ailment by associating the value or range of the quantified physical with a corresponding value or range of the library of threshold values or ranges. In some alternative or additional examples of operation 308, classifying the injury or ailment can be manually performed or otherwise implemented by a user, such by observing or otherwise assessing the value or range of the quantified physical characteristic to associate the value or range of the quantified physical with a corresponding value or range of the library of threshold values or ranges, or by observing or otherwise assessing collected, processed, or quantified multispectral imaging data according to other parameters or characteristics.

In some examples, the operation 308 can include tracking a change in the injury or ailment by comparing, using the processing circuitry, a value or range of the quantified physical characteristic to a historical value or range obtained by quantifying the physical characteristic of the biological tissue associated with the injury or ailment during at least one former point in time. For example, the mobile device can be in communication with an imaging database include a plurality of historical values or ranges based on a quantified physical characteristic of biological tissue associated with an injury or ailment of an individual patient at a previous or former point in time.

In such an example, a user can operate the mobile imaging system, such as via one or more user inputs to the user interface of the mobile device, to cause the processing circuity to quantify a difference between a value or range of the quantified physical characteristic and at least one historical value or range. In some alternative, or additional examples of operation 308, tracking a change in the injury or ailment by comparing a value or range of the quantified physical characteristic to a historical value or range can be manually performed, or otherwise implemented by a user, such by observing or otherwise assessing the value or range of the quantified physical characteristic to compare the value or range of the quantified physical characteristic with a at least one historical value or range, or observing or otherwise assessing collected, processed, or quantified multispectral imaging data according to other parameters or characteristics.

In some examples, the operation 308 can include comparing, using the processing circuitry, a value or range of the quantified physical characteristic to a historical value or range obtained by quantifying the physical characteristic of biological tissue associated with an injury or ailment of other patients. For example, the mobile device can be in communication with an imaging database containing at least one historical value or range based on a quantified physical characteristic of biological tissue associated with a similar injury or ailment of other patients collected at previous points in time. In such an example, a user can operate the mobile imaging system, such as via one or more user inputs to the user interface of the mobile device, to cause the processing circuity to quantify a difference between a value or range of the quantified physical characteristic and at least one historical value or range. In some alternative, or additional examples of operation 308, comparing a value or range of the quantified physical characteristic to a historical value or range can be manually performed, or otherwise implemented by a user, such by observing or otherwise assessing the value or range of the quantified physical characteristic to compare the value or range of the quantified physical characteristic with a at least one historical value or range, or observing or otherwise assessing collected, processed, or quantified multispectral imaging data according to other parameters or characteristics.

In any of the above examples of operation 308, the values or ranges discussed can be quantified physical characteristics of any of a deep tissue injury, an extent of tissue edema, tissue oxygenation, tissue inflammation due to infection, tissue perfusion, a wound bioburden or colonization estimate, or others. The discussed steps or operations can be performed in parallel or in a different sequence without materially impacting other operations. The method as discussed includes operations that can be performed by multiple different actors, devices, and/or systems. It is understood that subsets of the operations discussed in the method can be attributable to a single actor device, or system, and could be considered a separate standalone process or method.

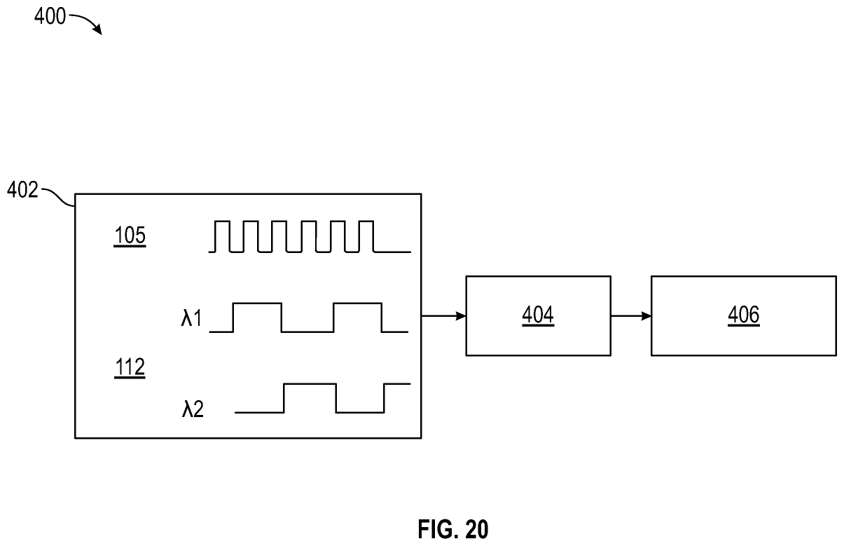

FIG. 20 illustrates flowchart of an example pathway 400 of recording various signals usable in a method of assessing an injury or ailment of a patient using an example imaging system 100 or 200. FIG. 20 is discussed with reference to the method 300 shown in FIG. 18. In some examples of the method 300, as discussed above, the operation 306 can include recording PPG signals, such as shown by FIG. 19.

As shown in FIG. 20, the box 402 can represent an example of a timing control configuration of an imaging system according to the present disclosure. In such an example, the illumination module 112 can be configured to emit light in a first wavelength (λ1) and in a second wavelength (λ2). In some examples, the illumination module 112 can be configured to activate a first group of light emitters and a second group of light emitters, such as any of the groups 191 of light emitters 113 shown in FIG. 13. In such an example, the illumination module 112 can be configured to perform or otherwise implement a cycle time of about, but not limited to, 50 milliseconds, such that the first wavelength of light (λ1) and the second wavelength of light (λ2) are repeatedly and alternatingly activated or otherwise emitted for a time period of 50 milliseconds. In some such examples, a camera system 105, such as including any of the first camera 106 (FIGS. 1-5) or the second camera 108 (FIGS. 1-5), can be configured to capture images at 20 frames per second; such as corresponding to 10 images per second for each of the first wavelength of light (λ1) and the second wavelength of light (λ1) emitted by the illumination module 112.

Such a timing control configuration can be helpful, for example, in stabilizing quantified values or ranges or calculated. In some examples, longer or shorter durations or configurations can be desirable and can performed or otherwise implemented, such as by configuring various operations of the camera system 105 or the illumination module 112. In some examples, the box 404 shown in FIG. 20 can represent any steps or operations of the operation 304 of the method 300 shown in, or described with regard to, FIG. 18. In some examples, the box 406 shown in FIG. 20 can represent any steps or operation of the operation 306 shown in, or described with regard to, FIG. 18.

Various examples of multispectral imaging data collected using an example of the imaging system 100 or 200 are shown in FIGS. 20-33 of U.S. Provisional Patent Application Ser. No. 63/042,957 entitled “MULTI-MODAL MOBILE THERMAL IMAGING SYSTEM” filed on Jun. 23, 2020, which is hereby incorporated by reference herein in its entirety. Accordingly, any of the devices, methods, or techniques describing in this document above may have been used, or can further be used to, collect and assess the multispectral imaging data (e.g. images) shown in FIGS. 20-33, or can be used to collect and assess similar multispectral imaging data of biological tissue of other patients associated with similar injuries or ailments.

In some examples, the imaging system 100 can help assess lymphedema using fluorescence imaging, such as shown in FIG. 20. In some examples, the imaging system 100 can help assess cellulitus, such as shown in FIGS. 21-22. In some examples, the imaging system 100 can help assess pseudocellulitis, such as shown in FIGS. 23-24. In some examples, the imaging system 100 can help assess the extent of infection, such as shown in FIGS. 25-26. In some examples, the imaging system 100 can help assess tissue perfusion or circulation, such as to help assess osteomyelitis, such as shown in FIGS. 27-29. In some examples, the imaging system 100 can filter out superficial surface tissue details or discoloration, such as to help assess a deeper wound. In some examples, the imaging system 100 can help assess a deep tissue injury (DTI), such as shown in FIGS. 30-31. In some examples, the imaging system 100 can help assess tissue health below a necrotic tissue/eschar, as shown in FIGS. 32-33. Still further uses of the imaging system 100 or 200 can include (1) measuring peripheral neuropathy via temperature, (2) point-of-care real-time fluorescence wound imaging to determine bacterial presence, location, and load, (3) transillumination, such as for the diagnosis of osteomyelitis in distal extremities such as toes, fingers, feet, and hands, and (4) spectroscopy, such as with or without the use of ICG or other fluorescent dyes, to map vascular distribution in distal limbs.

Of additional note to the present disclosure, the Center for Medicare and Medicaid Services (CMS) has issued Ambulatory Payment Classification (APC) code 5722, for the imaging for bacterial presence, location and load. The APC code is effective Jul. 1, 2020, and enables facility reimbursement under the Medicare Hospital Outpatient Prospective Payment System (OPPS). The 2020 hospital outpatient payment rate is $253.10 USD. This is accompanied by two category III (services and procedures using emerging technology) Common Procedural Technology (CPT) codes, which are also effective as of Jul. 1, 2020, and which enable physicians to request payment from payers for their work in providing the wound imaging procedure provided by the imaging system 100. The two CPT codes are 0598T, Noncontact real-time fluorescence wound imaging for bacterial presence, location, and load for a single site, and 0599T, would imaging of each additional anatomic site.

### EXAMPLES

The following non-limiting examples detail certain aspects of the present subject matter:

Example 1 is a mobile imaging system, comprising: a camera system configured to capture images in a visible, a near-infrared, and an infrared light spectrum, the camera system including a filter assembly including at least two optical filters; an illumination module activatable to emit light; a computer system including: processing circuitry configured to perform operations including: control the camera system to collect multispectral imaging data of biological tissue associated with an injury or ailment of a patient; and process the multispectral imaging data to assess the injury or ailment; a battery arranged to power the mobile imaging system; and a housing encompassing the processing circuitry and the battery, wherein the filter assembly and the illumination module are connected to the housing.

In Example 2, the subject matter of Example 1 includes, wherein the processing circuitry is configured to quantify a physical characteristic of biological tissue associated with the injury or ailment to assess the injury or ailment.

In Example 3, the subject matter of Example 2 includes, wherein processing circuitry is configured to compare a value or range of a quantified physical characteristic to library of threshold values or ranges, each defining a different class or category of the injury or ailment, to classify the injury or ailment to further assess the injury of ailment.

In Example 4, the subject matter of Examples 1-3 includes, wherein the computer system is a mobile phone including a user interface in communication with the processing circuitry, the user interface configured to output user instructions and receive user inputs to control the processing circuitry and the camera system.

In Example 5, the subject matter of Examples 1-4 includes, wherein each of the least two optical filters of the camera system includes one of a spectral filter, a bandpass filter, a dual-bandpass filter, a polarization filter, a visible filter, an ultraviolet filter, or a reference filter.

In Example 6, the subject matter of Examples 1-5 includes, wherein the camera system includes: a first camera configured to capture images in the visible and in the near-infrared light spectrums; and a second camera configured to capture images in the infrared light spectrum.

In Example 7, the subject matter of Example 6 includes, wherein the filter assembly includes: a base fixedly connected to the housing; and a filter member adjustably connected to the base and including the at least two optical filters, wherein the filter member is translatable or rotatable to sequentially position the at least two optical filters proximally to the first camera.

In Example 8, the subject matter of Example 7 includes, wherein the illumination module includes a controller configurable to control any of: a wavelength of the light to be emitted by the illumination module; a number of different wavelengths of light to be emitted; a cycle length of the illumination module, wherein the cycle length is a time period defined between activation and deactivation of the illumination module; and a cycle quantity of the illumination module, wherein the cycle quantity is a number of cycles the illumination module is configured to perform.

In Example 9, the subject matter of Examples 1-8 includes, wherein the illumination module is configured to sequentially emit light in at least three different wavelengths, and wherein the filter assembly includes at least three optical filters each configured for use during emission of one of the at least three wavelengths of light that the illumination module is configured to emit.

In Example 10, the subject matter of Example 9 includes, wherein the at least three different wavelengths are about 405, about 760, and about 850 nanometers.

Example 11 is a mobile imaging system, comprising: a camera system including: a first camera configured to capture images in a visible and in a near-infrared light spectrum; a second camera configured to capture images in an infrared light spectrum; a filter assembly including at least two optical filters selectively positionable with respect to the first camera; an illumination module including: a power supply; at least two groups of light emitters configured to emit light in different wavelengths; a computer system including: processing circuitry configured to perform operations including: control the camera system to collect a multispectral imaging data including a plurality of near-infrared and infrared images of biological tissue associated with an injury or ailment of a patient; and process the multispectral imaging data to assess the injury or ailment; a battery arranged to power the computer system and the camera system; and a housing encompassing the processing circuitry and the battery, wherein the filter assembly and the illumination module are connected to the housing.

In Example 12, the subject matter of Example 11 includes, wherein the filter assembly includes a mechanism synchronized with the illumination module and configured to sequentially position the at least two optical filters proximally to the first camera during emission of light in at least two different wavelengths.

In Example 13, the subject matter of Examples 11-12 includes, wherein the processing circuitry is configured to quantify a physical characteristic of biological tissue associated with the injury or ailment to assess the injury or ailment, and wherein the physical characteristic includes any of tissue edema or swelling, tissue oxygenation, tissue perfusion, bacterial load, bioburden, a wound area, or a wound volume.

In Example 14, the subject matter of Example 13 includes, wherein the processing circuitry is configured to compare a value or range of a quantified physical characteristic to a historical value or range based on the quantified physical characteristic of the biological tissue associated with the injury or ailment of the patient.

In Example 15, the subject matter of Examples 13-14 includes, wherein the processing circuitry is configured to compare a value or range of a quantified physical characteristic to a historical value or range based on a quantified physical characteristic of biological tissue associated with similar injuries or ailments of other patients.

Example 16 is a method of assessing an injury or ailment of a patient using a mobile imaging system; the method comprising: configuring the mobile imaging system based on the injury or ailment including configuring processing circuitry of a mobile phone arranged to control a near-infrared camera and an infrared camera; collecting multispectral imaging data of biological tissue associated with the injury or ailment, wherein the multispectral imaging data includes, at least a plurality of near-infrared and infrared images; and quantifying a physical characteristic of the biological tissue associated with the injury or ailment using the processing circuitry, wherein the physical characteristic includes at least one of tissue edema or swelling, tissue oxygenation, tissue perfusion, bacterial load, bioburden, a wound area, or a wound volume.

In Example 17, the subject matter of Example 16 includes, wherein collecting the multispectral imaging data includes sequentially positioning at least two optical filters in a position proximal to the near-infrared camera.

In Example 18, the subject matter of Example 17 includes, wherein collecting the multispectral imaging data includes sequentially illuminating the biological tissue with at least two wavelengths of light.

In Example 19, the subject matter of Examples 16-18 includes, wherein the method first comprises introducing fluorescent dye to the patient, and wherein collecting the multispectral imaging data includes collecting multispectral fluorescence imaging data.

In Example 20, the subject matter of Examples 16-19 includes, wherein the method further includes classifying the injury or ailment by comparing, using the processing circuitry, a value or range of the quantified physical characteristic to a library of threshold values or ranges, each defining a different class or category of the injury or ailment.

In Example 21, the subject matter of Examples 16-20 includes, wherein the method further includes tracking a change in the injury or ailment by comparing, using the processing circuitry, a value or range of the quantified physical characteristic to a historical value or range obtained by quantifying the physical characteristic of the biological tissue associated with the injury or ailment during at least one former point in time.

In Example 22, the subject matter of Examples 16-21 includes, wherein the method further includes comparing, using the processing circuitry, a value or range of the quantified physical characteristic to a historical value or range obtained by quantifying the physical characteristic of biological tissue associated with an injury or ailment of other patients.

Example 23 is at least one machine-readable medium including instructions that, when executed by processing circuitry, cause the processing circuitry to perform operations to implement of any of Examples 1-22.

Example 24 is an apparatus comprising means to implement of any of Examples 1-22.

Example 25 is a system to implement of any of Examples 1-22.

Example 26 is a method to implement of any of Examples 1-22.

Example 27 is a mobile imaging system, comprising: a camera system configured to capture images in a visible, a near-infrared, and an infrared light spectrum, the camera system including a filter assembly including at least two optical filters; an illumination module activatable to emit light; a computer system including processing circuitry including executable code configured to collect multispectral imaging data of biological tissue using the camera system, and to process the multispectral imaging data; a battery; and a housing.

In Example 28, the subject matter of Example 27 includes, wherein the processing circuitry is configured to quantify a physical characteristic of biological tissue associated with an injury or ailment.

In Example 29, the subject matter of Examples 27-28 includes, wherein processing circuitry is configured to compare a value or range of a quantified physical characteristic to a library of threshold values or ranges.

In Example 30, the subject matter of Examples 27-29 includes, wherein the computer system includes a mobile phone with a user interface configured to output user instructions and receive user inputs to control the system.

In Example 31, the subject matter of Examples 27-30 includes, wherein the at least two optical filters include one of a spectral filter, a bandpass filter, a dual-bandpass filter, a polarization filter, a visible filter, an ultraviolet filter, or a reference filter.

In Example 32, the subject matter of Examples 27-31 includes, wherein the camera system includes: a first camera configured to capture images in the visible and in the near-infrared light spectra; and a second camera configured to capture images in the infrared light spectrum.

In Example 33, the subject matter of Example 32 includes, wherein the filter assembly includes: a base fixedly connected to the housing; and a filter member adjustably connected to the base and including the at least two optical filters, wherein the filter member is translatable or rotatable to sequentially position the at least two optical filters proximally to the first camera.

In Example 34, the subject matter of Example 33 includes, wherein the illumination module includes a controller configurable to control any of: a wavelength of light to be emitted by the illumination module; a number of different wavelengths of light to be emitted; a cycle length of the illumination module, wherein the cycle length is a time period between activation and deactivation of the illumination module; and a cycle quantity of the illumination module, wherein the cycle quantity is a number of cycles the illumination module is configured to perform.

In Example 35, the subject matter of Examples 27-34 includes, wherein the illumination module is configured to sequentially emit light in at least three different wavelengths, and wherein the filter assembly includes at least three optical filters each configured for use during emission of one of the at least three different wavelengths of light that the illumination module is configured to emit.

In Example 36, the subject matter of Example 35 includes, wherein the at least three different wavelengths are about 405, about 760, and about 850 nanometers.

Example 37 is a mobile imaging system, comprising: a camera system including: a first camera configured to capture images in a visible and in a near-infrared light spectra; a second camera configured to capture images in an infrared light spectrum; a filter assembly including at least two optical filters selectively positionable with respect to the first camera; and an illumination module including at least two light emitters configured to emit light at different wavelengths; a computer system including processing circuitry configured to execute instructions to control the camera system to collect a multispectral imaging data including a plurality of near-infrared and infrared images of biological tissue and to process the multispectral imaging data; a battery; and a housing.

In Example 38, the subject matter of Example 37 includes, wherein the filter assembly includes a mechanism configured to sequentially position the at least two optical filters proximally to the first camera during emission of light in at least two different wavelengths.

In Example 39, the subject matter of Examples 37-38 includes, wherein the processing circuitry is configured to quantify a physical characteristic including any of tissue edema or swelling, tissue oxygenation, tissue perfusion, bacterial load, bioburden, a wound area, or a wound volume.

In Example 40, the subject matter of Examples 37-39 includes, wherein the processing circuitry is configured to compare a value or range of a quantified physical characteristic to a historical value or range.

In Example 41, the subject matter of Example 40 includes, wherein the value or range of the quantified physical characteristic is of biological tissue associated with injuries or ailments.

Example 42 is a method of assessing a medical condition of a patient using a mobile imaging system, comprising: configuring processing circuitry of a mobile phone to control a near-infrared camera and an infrared camera; collecting multispectral imaging data of biological tissue of the patient, wherein the multispectral imaging data includes, at least a plurality of near-infrared and infrared images; and quantifying a physical characteristic of the biological tissue including at least one of tissue edema or swelling, tissue oxygenation, tissue perfusion, bacterial load, bioburden, a wound area, or a wound volume.

In Example 43, the subject matter of Example 42 includes, wherein the collecting multispectral imaging data includes sequentially positioning at least two optical filters in a position proximal to the near-infrared camera.

In Example 44, the subject matter of Examples 42-43 includes, wherein the collecting the multispectral imaging data includes sequentially illuminating the biological tissue with at least two wavelengths of light.

In Example 45, the subject matter of Examples 42-44 includes, introducing fluorescent dye to the patient, and wherein the collecting the multispectral imaging data includes collecting multispectral fluorescence imaging data.

In Example 46, the subject matter of Examples 42-45 includes, classifying the injury or ailment by comparing, using the processing circuitry, a value or range of the quantified physical characteristic to a library of threshold values or ranges.

In Example 47, the subject matter of Examples 42-46 includes, tracking a change in the injury or ailment by comparing, using the processing circuitry, a value or range of the quantified physical characteristic to a historical value or range obtained by quantifying the physical characteristic of the biological tissue associated with the injury or ailment during at least one former point in time.

In Example 48, the subject matter of Examples 42-47 includes, comparing, using the processing circuitry, a value or range of the quantified physical characteristic to a historical value or range obtained by quantifying the physical characteristic of biological tissue associated with an injury or ailment of other patients.

Example 49 is at least one machine-readable medium including instructions that, when executed by processing circuitry, cause the processing circuitry to perform operations to implement of any of Examples 27-48.

Example 50 is an apparatus comprising means to implement of any of Examples 27-48.

Example 51 is a system to implement of any of Examples 27-48.

Example 52 is a method to implement of any of Examples 27-48.

Example 53 is a mobile imaging system, comprising: a camera system configured to capture images in a visible, a near-infrared, and an infrared light spectrum, the camera system including a filter assembly including at least two optical filters; an illumination module activatable to emit light; a computer system including processing circuitry including executable code configured to collect multispectral imaging data of biological tissue using the camera system, and to process the multispectral imaging data; a battery; and a housing.

In Example 54, the subject matter of Example 53 includes, wherein the processing circuitry is configured to quantify a physical characteristic of biological tissue associated with an injury or ailment.

In Example 55, the subject matter of Examples 53-54 includes, wherein processing circuitry is configured to compare a value or range of a quantified physical characteristic to a library of threshold values or ranges.

In Example 56, the subject matter of Examples 53-55 includes, wherein the computer system includes a mobile phone with a user interface configured to output user instructions and receive user inputs to control the system.

In Example 57, the subject matter of Examples 53-56 includes, wherein the at least two optical filters include one of a spectral filter, a bandpass filter, a dual-bandpass filter, a polarization filter, a visible filter, an ultraviolet filter, or a reference filter.

In Example 58, the subject matter of any of Examples 53-57 includes, wherein the camera system includes: a first camera configured to capture images in the visible and in the near-infrared light spectra; and a second camera configured to capture images in the infrared light spectrum.

In Example 59, the subject matter of Example 58 includes, wherein the filter assembly includes: a base fixedly connected to the housing; and a filter member adjustably connected to the base and including the at least two optical filters, wherein the filter member is translatable or rotatable to sequentially position the at least two optical filters proximally to the first camera.

In Example 60, the subject matter of Example 59 includes, wherein the illumination module includes a controller configurable to control any of: a wavelength of light to be emitted by the illumination module; a number of different wavelengths of light to be emitted; a cycle length of the illumination module, wherein the cycle length is a time period between activation and deactivation of the illumination module; and a cycle quantity of the illumination module, wherein the cycle quantity is a number of cycles the illumination module is configured to perform.

In Example 61, the subject matter of any of Examples 53-60 includes wherein the illumination module is configured to sequentially emit light in at least three different wavelengths, and wherein the filter assembly includes at least three optical filters each configured for use during emission of one of the at least three different wavelengths of light that the illumination module is configured to emit.

In Example 62, the subject matter of Example 61 includes, wherein the at least three different wavelengths are about 405, about 760, and about 850 nanometers.

In Example 63, the subject matter of any of Examples 53-62 includes, wherein the filter assembly includes a mechanism configured to sequentially position the at least two optical filters proximally to the first camera during emission of light in at least two different wavelengths.

Example 64 is a method of taking readings from a patient using a mobile imaging system, comprising: configuring processing circuitry of a mobile phone to control a near-infrared camera and an infrared camera; collecting multispectral imaging data of biological tissue of the patient, wherein the multispectral imaging data includes, at least a plurality of near-infrared and infrared images; and quantifying a physical characteristic of the biological tissue including at least one of tissue edema or swelling, tissue oxygenation, tissue perfusion, bacterial load, bioburden, a wound area, or a wound volume.

In Example 65, the subject matter of Example 64 includes, wherein the collecting multispectral imaging data includes sequentially positioning at least two optical filters in a position proximal to the near-infrared camera.

In Example 66, the subject matter of Examples 64-65 includes, wherein the collecting the multispectral imaging data includes sequentially illuminating the biological tissue with at least two wavelengths of light.

In Example 67, the subject matter of Examples 64-66 includes, introducing fluorescent dye to the patient, and wherein the collecting the multispectral imaging data includes collecting multispectral fluorescence imaging data.

Example 68 is at least one machine-readable medium including instructions that, when executed by processing circuitry, cause the processing circuitry to perform operations to implement of any of Examples 53-67.

Example 69 is an apparatus comprising means to implement of any of Examples 53-67.

Example 70 is a system to implement of any of Examples 53-67.

Example 71 is a method to implement of any of Examples 53-67.

In this document, the terms “a” or “an” are used, as is common in patent documents, to include one or more than one, independent of any other instances or usages of “at least one” or “one or more.”

The present detailed description includes references to the accompanying drawings, which form a part of the detailed description. The drawings show, by way of illustration, various embodiments in which the invention can be practiced. These embodiments are also referred to herein as “examples.” Such examples can include elements in addition to those shown or described.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

This description is intended to be illustrative, and not restrictive. The scope of the invention should be determined with reference to the appended claims, along with the full scope of equivalents to which such claims are entitled.

What is claimed is:

1. A mobile imaging system, comprising:
a camera system configured to capture images in a visible, a near-infrared, and an infrared light spectrum, the camera system including a filter assembly including at least two optical filters;
an illumination module activatable to emit light;
a computer system including processing circuitry including executable code configured to collect multispectral imaging data of biological tissue using the camera system, and to process the multispectral imaging data;
a battery; and
a housing.

2. The system of claim 1, wherein the processing circuitry is configured to quantify a physical characteristic of biological tissue associated with an injury or ailment.

3. The system of claim 1, wherein processing circuitry is configured to compare a value or range of a quantified physical characteristic to a library of threshold values or ranges.

4. The system of claim 1, wherein the computer system includes a mobile phone with a user interface configured to output user instructions and receive user inputs to control the system.

5. The system of claim 1, wherein the at least two optical filters include one of a spectral filter, a bandpass filter, a dual-bandpass filter, a polarization filter, a visible filter, an ultraviolet filter, or a reference filter.

6. The system of claim 1, wherein the camera system includes:
a first camera configured to capture images in the visible and in the near-infrared light spectra; and
a second camera configured to capture images in the infrared light spectrum.

7. The system of claim 6, wherein the filter assembly includes:
a base fixedly connected to the housing; and
a filter member adjustably connected to the base and including the at least two optical filters, wherein the filter member is translatable or rotatable to sequentially position the at least two optical filters proximally to the first camera.

8. The system of claim 7, wherein the illumination module includes a controller configurable to control any of:
a wavelength of light to be emitted by the illumination module;
a number of different wavelengths of light to be emitted;
a cycle length of the illumination module, wherein the cycle length is a time period between activation and deactivation of the illumination module; and
a cycle quantity of the illumination module, wherein the cycle quantity is a number of cycles the illumination module is configured to perform.

9. The system of claim 1, wherein the illumination module is configured to sequentially emit light in at least three different wavelengths, and wherein the filter assembly includes at least three optical filters each configured for use during emission of one of the at least three different wavelengths of light that the illumination module is configured to emit.

10. The system of claim 9, wherein the at least three different wavelengths are about 405, about 760, and about 850 nanometers.

11. A mobile imaging system, comprising:
a camera system including:
a first camera configured to capture images in a visible and in a near-infrared light spectra;
a second camera configured to capture images in an infrared light spectrum;
a filter assembly including at least two optical filters selectively positionable with respect to the first camera; and
an illumination module including at least two light emitters configured to emit light at different wavelengths;
a computer system including processing circuitry configured to execute instructions to control the camera system to collect a multispectral imaging data including a plurality of near-infrared and infrared images of biological tissue and to process the multispectral imaging data;
a battery; and
a housing.

12. The system of claim 11, wherein the filter assembly includes a mechanism configured to sequentially position the at least two optical filters proximally to the first camera during emission of light in at least two different wavelengths.

13. The system of claim 11, wherein the processing circuitry is configured to quantify a physical characteristic including any of tissue edema or swelling, tissue oxygenation, tissue perfusion, bacterial load, bioburden, a wound area, or a wound volume.

14. The system of claim 11, wherein the processing circuitry is configured to compare a value or range of a quantified physical characteristic to a historical value or range.

15. The system of claim 14, wherein the value or range of the quantified physical characteristic is of biological tissue associated with injuries or ailments.

16. A method of assessing a medical condition of a patient using a mobile imaging system, comprising:
configuring processing circuitry of a mobile phone to control a near-infrared camera and an infrared camera;
collecting multispectral imaging data of biological tissue of the patient, wherein the multispectral imaging data includes at least a plurality of near-infrared and infrared images; and
quantifying a physical characteristic of the biological tissue including at least one of tissue edema or swelling, tissue oxygenation, tissue perfusion, bacterial load, bioburden, a wound area, or a wound volume.

17. The method of claim 16, wherein the collecting multispectral imaging data includes sequentially positioning at least two optical filters in a position proximal to the near-infrared camera.

18. The method of claim 16, wherein the collecting the multispectral imaging data includes sequentially illuminating the biological tissue with at least two wavelengths of light.

19. The method of claim 16, further comprising introducing fluorescent dye to the patient, and wherein the collecting the multispectral imaging data includes collecting multispectral fluorescence imaging data.

20. The method of claim 16, further comprising classifying the injury or ailment by comparing, using the processing circuitry, a value or range of the quantified physical characteristic to a library of threshold values or ranges.

21. The method of claim 16, further comprising tracking a change in the injury or ailment by comparing, using the processing circuitry, a value or range of the quantified physical characteristic to a historical value or range obtained by quantifying the physical characteristic of the biological tissue associated with the injury or ailment during at least one former point in time.

22. The method of claim 16, further comprising comparing, using the processing circuitry, a value or range of the quantified physical characteristic to a historical value or range obtained by quantifying the physical characteristic of biological tissue associated with an injury or ailment of other patients.
